(19) United States
(12) Patent Application Publication
(10) Pub. No.: US 2021/0060230 A1
(43) Pub. Date: Mar. 4, 2021

(54) THERMAL CONTROL SYSTEM

(71) Applicant: Stryker Corporation, Kalamazoo, MI (US)

(72) Inventors: Christopher John Hopper, Kalamazoo, MI (US); Gregory S. Taylor, Kalamazoo, MI (US)

(21) Appl. No.: 16/957,551

(22) PCT Filed: Dec. 26, 2017
(86) PCT No.: PCT/US2018/064685

§ 371 Date: Jun. 24, 2020

Related U.S. Application Data
(60) Provisional application No. 62/610,334, filed on Dec. 26, 2017.

(51) Int. Cl. A61M 1/36; A61F 7/00; A61F 7/08; A61B 5/145; A61B 5/00; A61B 5/01

(52) U.S. Cl. CPC A61M 1/369; A61F 7/0085; A61F 7/08; A61B 5/14542; A61B 5/6866; A61M 2230/20; A61B 5/01; A61F 2007/0056; A61F 2007/0096; A61M 2205/502; A61M 2230/50; A61B 5/4872

(57) ABSTRACT

A thermal control system for controlling a temperature of a fluid delivered to a patient is provided. The system includes a thermal control unit having a fluid inlet and outlet, a circulation channel, a pump, a heat exchanger, a fluid temperature sensor and a controller that controls the heat exchanger in order to automatically bring a patients temperature to a target temperature. In some embodiments, the control unit includes a user interface adapted to receive a non-temperature patient parameter (e.g. BMI) that the controller uses, along with patient core temperature readings, to control the heat exchanger. The controller may also or alternatively control the heat exchanger based on both core and peripheral patient temperature readings. An auxiliary thermal therapy device for controlling a temperature of the patients blood, air breathed by the patient, and/or other fluid, may also be controlled by the thermal control unit.

### CROSS-REFERENCE TO RELATED APPLICATIONS

This application claims priority to U.S. provisional patent application Ser. No. 62/610,334 filed Dec. 26, 2017, by inventors Christopher John Hopper et al. and entitled THERMAL CONTROL SYSTEM, the complete disclosure of which is incorporated herein by reference.

### BACKGROUND

The present disclosure relates to a thermal control system for controlling the temperature of a patient by delivering one or more temperature-controlled fluids to the patient and/or to thermal pads positioned in contact with the patient.

Thermal control systems are known in the art for controlling the temperature of a patient by supplying temperature-controlled fluid to one or more pads (or similar structures) that are positioned in contact with, or adjacent to, a patient. The temperature of the fluid is controlled by a thermal control unit that provides fluid to the pads. After passing through the pads, the fluid is returned to the control unit where any necessary adjustments to the returning fluid temperature are made before being pumped back to the pads. In some instances, the temperature of the fluid is controlled to a target fluid temperature, while in other instances the temperature of the fluid is automatically controlled in order to effectuate a target patient temperature. When controlling a patient's temperature, a patient temperature probe may be attached to the control unit in order to provide patient temperature readings as feedback to the control unit so that it can make the necessary temperature adjustments to the circulating fluid.

### SUMMARY

The present disclosure provides various improved aspects to a thermal control system. In one embodiment, the present disclosure includes a thermal control unit that takes into account additional factors besides a patient's core temperature when controlling the temperature of the fluid delivered to the thermal pads. Such additional factors, which may include the patient's peripheral temperature, BMI, and/or other factors, allow the thermal control unit to reduce temperature overshoot, achieve the target patient temperature more quickly, and/or reduce thermal stresses upon the patient. Other aspects of the present disclosure include using the thermal control unit in conjunction with an auxiliary thermal therapy device that controls the patient's temperature using one or more non-thermal pad structures, such as a esophageal heat transfer device, an air temperature controller, and/or an extracorporeal blood thermal transfer device. In still other aspects, the extracorporeal blood thermal transfer device may be incorporated into the thermal control unit such that it controls both a temperature of fluid supplied to the thermal pads and a temperature of blood received from, and returned to, the patient.

According to a first embodiment of the present disclosure, a thermal control unit for controlling a patient's temperature is provided that includes a fluid outlet, a fluid inlet, a fluid circulation path, a pump, a blood inlet, a blood outlet, a blood circulation path, first and second heat exchangers, and a controller. The fluid outlet and inlet are adapted to couple to fluid supply and return lines, respectively. The pump circulates fluid through the fluid circulation path from the fluid inlet to the fluid outlet. The blood inlet is adapted to receive blood from the patient and the blood outlet is adapted to return blood to the patient. The blood circulation path is coupled to the blood outlet and the blood inlet. The first heat exchanger is adapted to add or remove heat from the fluid circulating through the fluid circulation path, and the second heat exchanger is adapted to add or remove heat from the blood circulating through the blood circulation path. The controller communicates with the first and second heat exchangers and controls a temperature of the fluid and a temperature of the blood in order to bring the patient to a target patient temperature.

According to other aspects of the present disclosure, the controller independently controls the fluid temperature to a target fluid temperature and the blood temperature to a target blood temperature. The target fluid temperature and the target blood temperature may vary from each other at different times during a thermal therapy session.

In some embodiments, the thermal control unit further includes a core temperature probe port and a peripheral temperature probe port. The core temperature probe port is adapted to receive a core temperature probe for measuring a core temperature of the patient, and the peripheral temperature probe port is adapted to receive a peripheral temperature probe for measuring a peripheral temperature of the patient. The controller controls the temperature of both the fluid and the blood based on temperature readings received from both the core temperature probe port and the peripheral temperature probe port.

The controller, in at least one embodiment, controls the second heat exchanger based upon a temperature of the blood in the blood circulation path. The controller may also control the first heat exchanger based upon a temperature of both the blood and the fluid in the fluid circulating path.

In some embodiments, the fluid circulation path and blood circulation path are both within a cartridge adapted to be inserted into and out of a chamber defined in the thermal control unit. The thermal control unit may be constructed such that the first and second heat exchangers are fluidly isolated from the cartridge and remain within the thermal control unit when the cartridge is removed.

The controller, in some embodiments, is adapted to infer a patient peripheral temperature at a location adjacent a thermal pad based upon a heat transfer rate between the patient and the thermal pad. The thermal pad is fluidly coupled to the fluid outlet and fluid inlet and the controller is further adapted to control the first and second heat exchangers based on the inferred patient peripheral temperature.

A user interface is included that may be configured to receive a non-temperature patient parameter. The controller controls the first and second heat exchangers based partially on the non-temperature patient parameter. The non-temperature patient parameter may be a body mass index (BMI), a body surface area (BSA), a patient weight, a patient height, or a similar parameter.

A first intravenous needle may be coupled to the blood inlet along with a second intravenous needle coupled to the blood outlet. Each of the first and second intravenous needles are adapted to be inserted into a peripheral vein of the patient and thereby allow blood to circulate from the patient through the thermal control unit and back to the patient.

One or more sensors may be included with the thermal control unit that are positioned therein and that detect a vital sign of the patient from the blood flowing through the blood circulation path. The vital sign may be a blood pressure, an oxygenation level of the patient's blood, and/or another parameter.

According to another embodiment, a thermal control unit controlling a patient's temperature is provided that includes a fluid outlet, a fluid inlet, a fluid circulation channel, a pump, a heat exchanger, a fluid temperature sensor, a patient temperature probe port, a user interface, and a controller. The fluid outlet and inlet are adapted to couple to fluid supply and return lines, respectively, that supply fluid, and receive fluid from, a thermal pad. The thermal pad is adapted to be wrapped around a portion of a patient's body. The pump circulates fluid through the fluid circulation channel from the fluid inlet to the fluid outlet. The heat exchanger is adapted to add or remove heat from the fluid circulating through the fluid circulation channel. The fluid temperature sensor senses a temperature of the fluid. The patient temperature probe port receives patient core temperature readings from a patient temperature probe. The user interface receives a non-temperature patient parameter, and the controller communicates with the patient temperature probe port, the pump, the fluid temperature sensor, and the user interface. The controller is also adapted to control the heat exchanger based on both the patient core temperature readings and the non-temperature patient parameter.

According to other aspects of the disclosure, the controller controls the heat exchanger in order to automatically bring a temperature of the patient to a target patient temperature.

The non-temperature patient parameter may be one of a body mass index (BMI), a body surface area (BSA), a patient weight, a patient height, or the like.

In some embodiments, the thermal control unit further includes a patient peripheral temperature port adapted to receive patient peripheral temperature readings from a peripheral patient temperature sensor that measures a peripheral temperature of the patient. The controller is further adapted to control the heat exchanger based on the patient peripheral temperature readings. In some embodiments, the controller controls the heat exchanger based on differences between the patient core temperature readings and the patient peripheral temperature readings.

The thermal control unit may further include a second fluid outlet and a second fluid inlet. The second fluid outlet is adapted to couple to a fluid supply line of an auxiliary thermal therapy device and the second fluid inlet is adapted to couple to a fluid return line of the auxiliary thermal therapy device. The auxiliary thermal therapy device is adapted to add or remove heat from the patient, and is a type of device that is different from a thermal pad.

In some embodiments, the auxiliary thermal therapy device includes a fluid passageway positioned in the patient's esophagus that receives circulating fluid from the second fluid outlet.

The second fluid outlet and second fluid inlet may be fluidly coupled to each other via a second circulation channel that is fluidly isolated from the circulation channel. In such embodiments, the second circulation channel may be adapted to receive blood from the patient and the auxiliary thermal therapy device may include a first intravenous needle and a second intravenous needle. The first intravenous needle is coupled to the second fluid outlet and the second intravenous needle is coupled to the second fluid inlet. Each of the first and second intravenous needles is adapted to be inserted into a peripheral vein of the patient.

In other embodiments, the auxiliary thermal therapy device is adapted to deliver temperature-controlled air to be breathed in by the patient. In such embodiments, an air temperature controller may include an air channel and a second heat exchanger adapted to control a temperature of air passing through the air channel. The air channel fluidly communicates with the second fluid outlet in order to supply temperature-controlled air to the auxiliary thermal therapy device.

According to another embodiment of the present disclosure, a thermal control unit for controlling a patient's temperature is provided that includes a fluid outlet, a fluid inlet, a circulation channel, a pump, a heat exchanger, a fluid temperature sensor, a patient core temperature probe port, a patient peripheral temperature probe port, and a controller. The fluid outlet and inlet are adapted to couple to fluid supply and return lines, respectively, that supply fluid, and receive fluid from, a thermal pad. The thermal pad is adapted to be wrapped around a portion of the patient's body. The pump circulates fluid through the circulation channel from the fluid inlet to the fluid outlet. The heat exchanger adds or removes heat from the fluid circulating in the circulation channel. The fluid temperature sensor senses a temperature of the fluid. The patient core temperature probe port is adapted to receive patient core temperature readings from a patient temperature probe, and the patient peripheral temperature probe port is adapted to receive patient peripheral temperature readings from a peripheral temperature sensor. The controller communicates with the patient core temperature probe port, the patient peripheral temperature probe port, the pump, and the fluid temperature sensor. The controller controls the heat exchanger based on both the patient core temperature readings and the patient peripheral temperature reading, and the controller controls the heat exchanger in order to automatically bring the patient core temperature readings to a target patient temperature.

According to other aspects, the controller controls the heat exchanger based on differences between the patient core temperature readings and the patient peripheral temperature readings. In some embodiments, the controller automatically controls a temperature of the fluid in order to prevent the differences from exceeding a predetermined maximum.

The thermal control unit may also include a transceiver adapted to communicate with an auxiliary thermal therapy device that supplies heat to, and removes heat from, the patient.

According to another embodiment of the present disclosure, a thermal control unit for controlling a patient's temperature is provided that includes a fluid outlet, a fluid inlet, a circulation channel, a pump, a heat exchanger, a fluid temperature sensor, a patient core temperature probe port, a transceiver, and a controller. The fluid outlet and inlet are adapted to couple to fluid supply and return lines, respectively, that supply fluid, and receive fluid from, a thermal pad. The thermal pad is adapted to be wrapped around a portion of the patient's body. The pump circulates fluid through the circulation channel from the fluid inlet to the fluid outlet. The heat exchanger adds or removes heat from the fluid circulating in the circulation channel. The fluid temperature sensor senses a temperature of the fluid. The patient core temperature probe port is adapted to receive patient core temperature readings from a patient temperature probe. The transceiver communicates with an auxiliary thermal therapy device that is of a type other than a thermal pad. The auxiliary thermal therapy device is adapted to add or remove heat from the patient. The controller communicates with the patient core temperature probe port, the pump, and the fluid temperature sensor. The controller is adapted to control the heat exchanger based on both the patient core temperature readings and information received from the auxiliary thermal therapy device.

According to other aspects, the auxiliary thermal therapy device is adapted to deliver temperature-controlled air to be breathed in by the patient, or temperature-controlled blood to the patient, or temperature-controlled liquid to the esophagus of the patient.

The information received from the auxiliary thermal therapy device, in some embodiments, includes any one or more of the following: a temperature of fluid delivered to the patient via the auxiliary thermal therapy device; a patient temperature measured at a location on the patient different from a location of the patient temperature probe; and/or a quantity of heat added to or removed from the patient via the auxiliary thermal therapy device.

In some embodiments, the auxiliary thermal therapy device is adapted to add or remove heat from the patient by delivering temperature-controlled auxiliary fluid to the patient. The temperature-controlled auxiliary fluid may be blood, gas, or any other fluid circulating within the thermal control unit that is fluidly isolated from the circulation channel.

In some embodiments, the controller is adapted to control a first target temperature for the auxiliary fluid and a second target temperature for the circulating fluid. The first and second target temperatures may differ from each other during a patient's thermal therapy session.

According to another embodiment of the present disclosure, a thermal control unit for controlling a patient's temperature is provided that includes a fluid outlet, a fluid inlet, an auxiliary fluid outlet, an auxiliary fluid inlet, a circulation channel, a pump, a heat exchanger, a fluid temperature sensor, a patient core temperature probe port, and a controller. The fluid outlet and inlet are adapted to couple to fluid supply and return lines, respectively, that supply fluid, and receive fluid from, a thermal pad. The thermal pad is adapted to be wrapped around a portion of the patient's body. The auxiliary fluid outlet and auxiliary fluid inlet are adapted to couple to fluid supply and fluid return lines, respectively, of an auxiliary thermal therapy device. The auxiliary thermal therapy device is adapted to add or remove heat from the patient and is a type of thermal therapy device different from a thermal pad. The pump circulates fluid through the circulation channel from the fluid inlet to the fluid outlet. The heat exchanger adds or removes heat from the fluid circulating in the circulation channel. The fluid temperature sensor senses a temperature of the fluid. The patient core temperature probe port is adapted to receive patient core temperature readings from a patient temperature probe. The controller communicates with the patient core temperature probe port, the pump, and the fluid temperature sensor. The controller is adapted to set a first target temperature for fluid delivered to the fluid outlet and to set a second target temperature for fluid delivered to the auxiliary fluid outlet. The first and second target temperatures differ from each other at least once during a patient's thermal therapy session.

According to other aspects, the controller controls the first and second target temperatures in order to automatically bring a temperature of the patient to a target patient temperature.

In some embodiments, the auxiliary fluid outlet and auxiliary fluid inlet are in fluid communication with an auxiliary fluid channel, and the auxiliary fluid channel is fluidly isolated from the circulation channel. In other embodiments, the auxiliary fluid is not fluidly isolated from the circulation channel.

According to another embodiment of the present disclosure, a thermal control unit for controlling a patient's temperature is provided. The thermal control unit includes a fluid outlet, a fluid inlet, a circulation channel, a pump, a heat exchanger, a first fluid temperature sensor, a second fluid temperature sensor, a patient core temperature probe port, and a controller. The fluid outlet and inlet are adapted to couple to fluid supply and return lines, respectively, that supply fluid to, and receive fluid from, a thermal pad. The thermal pad is adapted to be wrapped around a portion of the patient's body. The pump circulates fluid through the circulation channel from the fluid inlet to the fluid outlet. The heat exchanger adds or removes heat from the fluid circulating in the circulation channel. The first fluid temperature sensor senses a temperature of the fluid delivered to the thermal pad, and the second fluid temperature sensor senses a temperature of the fluid returning from the thermal pad. The patient core temperature probe port is adapted to receive patient core temperature readings from a patient temperature probe. The controller communicates with the patient core temperature probe port, the pump, and the first and second fluid temperature sensors. The controller infers a patient peripheral temperature at a location adjacent the thermal pad based upon a difference between temperature readings from the first and second fluid temperature sensors. The controller is also adapted to control the heat exchanger based on both the patient core temperature readings and the inferred patient peripheral temperature.

According to other aspects, the controller infers the patient peripheral temperature by calculating a heat transfer rate between the patient and the thermal pad.

In some embodiments, the thermal control unit further includes a second fluid outlet and a second fluid inlet. The second fluid outlet is adapted to fluidly couple to a fluid supply line of an auxiliary thermal therapy device. The auxiliary thermal therapy device is of a type other than a thermal pad and is adapted to add or remove heat from the patient. The second fluid inlet is adapted to couple to a fluid return line of the auxiliary thermal therapy device. The controller controls the auxiliary thermal therapy device in order to bring a temperature of the patient to a target patient temperature.

The auxiliary thermal therapy device is constructed to deliver temperature-controlled air to be breathed in by the patient, or to deliver temperature-controlled liquid to the patient's esophagus, or is constructed in other manners.

Before the various embodiments disclosed herein are explained in detail, it is to be understood that the claims are not to be limited to the details of operation or to the details of construction, nor to the arrangement of the components set forth in the following description or illustrated in the drawings. The embodiments described herein are capable of being practiced or being carried out in alternative ways not expressly disclosed herein. Also, it is to be understood that the phraseology and terminology used herein are for the purpose of description and should not be regarded as limiting. The use of “including” and “comprising” and variations thereof is meant to encompass the items listed thereafter and equivalents thereof as well as additional items and equivalents thereof. Further, enumeration may be used in the description of various embodiments. Unless otherwise expressly stated, the use of enumeration should not be construed as limiting the claims to any specific order or number of components. Nor should the use of enumeration be construed as excluding from the scope of the claims any additional steps or components that might be combined with or into the enumerated steps or components.

### BRIEF DESCRIPTION OF THE DRAWINGS

FIG. 1 is a perspective view of a first embodiment of a thermal control system according to the present disclosure that may be used to provide thermal treatment to a patient;

FIG. 2 is a block diagram of the thermal control system of FIG. 1;

FIG. 3 is an illustrative control loop diagram that may be incorporated into any of the embodiments of the thermal control units disclosed herein;

FIG. 4 is a block diagram of a second embodiment of a thermal control system according to the present disclosure that may be used to provide thermal treatment to a patient;

FIG. 5 is a block diagram of a third embodiment of a thermal control system according to the present disclosure that may be used to provide thermal treatment to a patient;

FIG. 6 is a perspective view of a first manner of using the thermal control system of FIG. 5 to provide thermal treatment to a patient;

FIG. 7 is a perspective view of a second manner of using the thermal control system of FIG. 5 to provide thermal treatment to a patient;

FIG. 8 is a block diagram of a fourth embodiment of a thermal control system according to the present disclosure that may be used to provide thermal treatment to a patient;

FIG. 9 is an elevation view of a cartridge usable with the thermal control system of FIG. 8;

FIG. 10 is a block diagram of internal flow channels in one embodiment of the cartridge of FIG. 9; and

FIG. 11 is a graph of patient core temperature, patient subcutaneous temperature, fluid temperature, and blood temperature illustrating an example of a patient being cooled with the thermal control system of FIG. 8.

### DETAILED DESCRIPTION OF THE EMBODIMENTS

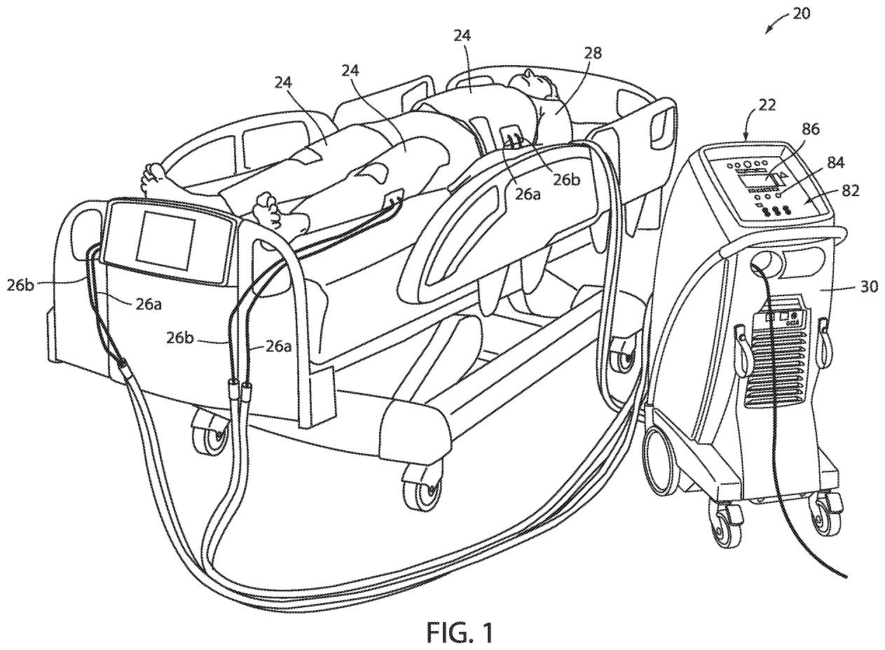

A thermal control system 20 according to one embodiment of the present disclosure is shown in FIG. 1. Thermal control system 20 is adapted to control the temperature of a patient 28, which may involve raising, lowering, and/or maintaining the patient's temperature. Thermal control system 20 includes a thermal control unit 22 coupled to one or more thermal therapy devices 24. The thermal therapy devices 24 are illustrated in FIG. 1 to be thermal pads, but it will be understood that thermal therapy devices 24 may take on other forms, such as, but not limited to, blankets, vests, patches, caps, catheters, or other structures that receive temperature-controlled fluid. For purposes of the following written description, thermal therapy devices 24 will be referred to as thermal pads 24, but it will be understood by those skilled in the art that this terminology is used merely for convenience and that the phrase “thermal pad” is intended to cover all of the different variations of thermal therapy devices 24 mentioned above (e.g. blankets, vests, patches, caps, catheters, etc.) and variations thereof.

Thermal control unit 22 is coupled to thermal pads 24 via a plurality of hoses 26. Thermal control unit 22 delivers temperature-controlled fluid (such as, but not limited to, water or a water mixture) to the thermal pads 24 via the fluid supply hoses 26a. After the temperature-controlled fluid has passed through thermal pads 24, thermal control unit 22 receives the temperature-controlled fluid back from thermal pads 24 via the return hoses 26b.

In the embodiment of thermal control system 20 shown in FIG. 1, three thermal pads 24 are used in the treatment of patient 28. A first thermal pad 24 is wrapped around a patient's torso, while second and third thermal pads 24 are wrapped, respectively, around the patient's right and left legs. Other configurations can be used and different numbers of thermal pads 24 may be used with thermal control unit 22, depending upon the number of inlet and outlet ports that are included with thermal control unit 22. By controlling the temperature of the fluid delivered to thermal pads 24 via supply hoses 26a, the temperature of the patient 28 can be controlled via the close contact of the pads 24 with the patient 28 and the resultant heat transfer therebetween.

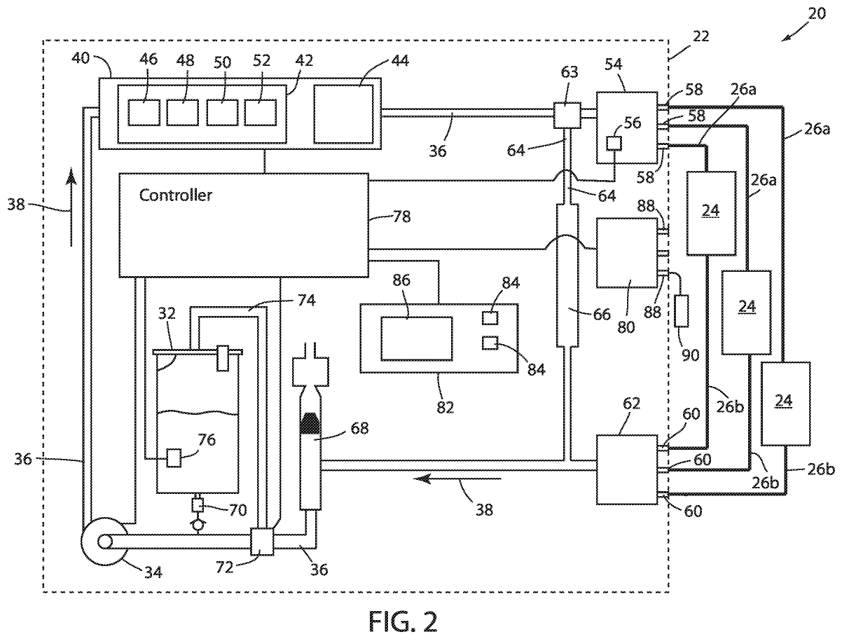

As shown in FIG. 2, thermal control unit 22 includes a main body 30 to which a removable reservoir 32 may be coupled and uncoupled. Removable reservoir 32 is configured to hold the fluid that is to be circulated through control unit 22 and the one or more thermal pads 24. By being removable from thermal control unit 22, reservoir 32 can be easily carried to a sink or faucet for filling and/or dumping of the water or other fluid. This allows users of thermal control system 20 to more easily fill control unit 22 prior to its use, as well as to drain unit 22 after use.

Thermal control unit 22 also includes a pump 34 for circulating fluid through a circulation channel 36. Pump 34, when activated, circulates the fluid through circulation channel 36 in the direction of arrows 38 (clockwise in FIG. 2). Starting at pump 34 the circulating fluid first passes through a heat exchanger 40 that adjusts, as necessary, the temperature of the circulating fluid. Heat exchanger 40 may take on a variety of different forms. In some embodiments, heat exchanger 40 is a thermoelectric heater and cooler. In the embodiment shown in FIG. 2, heat exchanger 40 includes a chiller 42 and a heater 44. Further, in the embodiment shown in FIG. 2, chiller 42 is a conventional vapor-compression refrigeration unit having a compressor 46, a condenser 48, an evaporator 50, an expansion valve (not shown), and a fan 52 for removing heat from the compressor 46. Other types of chillers and/or heaters may be used.

After passing through heat exchanger 40, the circulating fluid is delivered to an outlet manifold 54 having an outlet temperature sensor 56 and a plurality of outlet ports 58. Temperature sensor 56 is adapted to detect a temperature of the fluid inside of outlet manifold 54 and report it to a controller 78. Outlet ports 58 are coupled to supply hoses 26a. Supply hoses 26a are coupled, in turn, to thermal pads 24 and deliver temperature-controlled fluid to the thermal pads 24. The temperature-controlled fluid, after passing through the thermal pads 24, is returned to thermal control unit 22 via return hoses 26b. Return hoses 26b couple to a plurality of inlet ports 60. Inlet ports 60 are fluidly coupled to an inlet manifold 62 inside of thermal control unit 22.

Control unit 22 also includes a bypass line 64 fluidly coupled to outlet manifold 54 and inlet manifold 62 (FIG. 2). Bypass line 64 allows fluid to circulate through circulation channel 36 even in the absence of any thermal pads 24 or hoses 26a being coupled to any of outlet ports 58. In the illustrated embodiment, bypass line 64 includes an optional filter 66 that is adapted to filter the circulating fluid. If included, filter 66 may be a particle filter adapted to filter out particles within the circulating fluid that exceed a size threshold, or filter 66 may be a biological filter adapted to purify or sanitize the circulating fluid, or it may be a combination of both. In some embodiments, filter 66 is constructed and/or positioned within thermal control unit 22 in any of the manners disclosed in commonly assigned U.S. patent application Ser. No. 62/404,676 filed Oct. 11, 2016, by inventors Marko Kostic et al. and entitled THERMAL CONTROL SYSTEM, the complete disclosure of which is incorporated herein by reference.

The flow of fluid through bypass line 64 is controllable by way of a bypass valve 63 positioned at the intersection of bypass line 64 and outlet manifold 54 (FIG. 2). When open, bypass valve 63 allows fluid to flow through circulation channel 36 to outlet manifold 54, and from outlet manifold 54 to the connected thermal pads 24. When closed, bypass valve 63 stops fluid from flowing to outlet manifold 54 (and thermal pads 24) and instead diverts the fluid flow along bypass line 64. In some embodiments, bypass valve 63 may be controllable such that selective portions of the fluid are directed to outlet manifold 54 and along bypass line 64. The stopping of fluid flow to thermal pads 24 via bypass valve 63 may occur during the thermal treatment of a patient, as well as at other times.

The incoming fluid flowing into inlet manifold 62 from inlet ports 60 and/or bypass line 64 travels back toward pump 34 and into an air remover 68. Air remover 68 includes any structure in which the flow of fluid slows down sufficiently to allow air bubbles contained within the circulating fluid to float upwardly and escape to the ambient surroundings. In some embodiments, air remover 68 is constructed in accordance with any of the configurations disclosed in commonly assigned U.S. patent application Ser. No. 15/646,847 filed Jul. 11, 2017, by inventor Gregory S. Taylor and entitled THERMAL CONTROL SYSTEM, the complete disclosure of which is hereby incorporated herein by reference. After passing through air remover 68, the circulating fluid flows past a valve 70 positioned beneath fluid reservoir 32. Fluid reservoir 32 supplies fluid to thermal control unit 22 and circulation channel 36 via valve 70, which may be a conventional check valve, or other type of valve, that automatically opens when reservoir 32 is coupled to thermal control unit 22 and that automatically closes when reservoir 32 is decoupled from thermal control unit 22 (see FIG. 2). After passing by valve 70, the circulating fluid travels to pump 34 and the circuit is repeated.

In the embodiment shown in FIG. 2, thermal control unit 22 also includes a reservoir valve 72 that is adapted to selectively move fluid reservoir 32 into and out of line with circulation channel 36. Reservoir valve 72 is positioned in circulation channel 36 between air remover 68 and valve 70, although it will be understood that reservoir valve 72 may be moved to different locations within circulation channel 36. Reservoir valve 72 is coupled to circulation channel 36 as well as a reservoir channel 74. When reservoir valve 72 is open, fluid from air remover 68 flows along circulation channel 36 to pump 34 without passing through reservoir 32 and without any fluid flowing along reservoir channel 74. When reservoir valve 72 is closed, fluid coming from air remover 68 flows along reservoir channel 74, which feeds the fluid into reservoir 32. Fluid inside of reservoir 32 then flows back into circulation channel 36 via valve 70. Once back in circulation channel 36, the fluid flows to pump 34 and is pumped to the rest of circulation channel 36 and thermal pads 24 and/or bypass line 64. In some embodiments, reservoir valve 72 is either fully open or fully closed, while in other embodiments, reservoir valve 72 may be partially open or partially closed. In either case, reservoir valve 72 is under the control of controller 78.

Thermal control unit 22 also includes a reservoir temperature sensor 76. Reservoir temperature sensor 76 reports its temperature readings to controller 78. When reservoir valve 72 is open, the fluid inside of reservoir 32 stays inside of reservoir 32 (after the initial drainage of the amount of fluid needed to fill circulation channel 36 and thermal pads 24). This residual fluid is substantially not affected by the temperature changes made to the fluid within circulation channel 36 as long as reservoir valve 72 remains open. This is because the residual fluid that remains inside of reservoir 32 after circulation channel 36 and thermal pads 24 have been filled does not pass through heat exchanger 40 and remains substantially thermally isolated from the circulating fluid. Two results flow from this: first, heat exchanger 40 does not need to expend energy on changing the temperature of the residual fluid in reservoir 32, and second, the temperature of the circulating fluid in circulation channel 36 will deviate from the temperature of the residual fluid as the circulating fluid circulates through heat exchanger 40.

Controller 78 utilizes a temperature control algorithm to control reservoir valve 72 that, in some embodiments, includes the same temperature control algorithm 160 disclosed in commonly assigned U.S. patent application Ser. No. 62/577,772, filed on Oct. 27, 2017, by inventors Gregory Taylor et al. and entitled THERMAL SYSTEM WITH MEDICATION INTERACTION, the complete disclosure of which is incorporated herein by reference. In other embodiments, controller 78 utilizes a different control algorithm. In still other embodiments, thermal control unit 22 is modified to omit reservoir valve 72, reservoir channel 74, and/or reservoir temperature sensor 76. Thermal control unit 22 may also be modified such that reservoir 32 is always in the path of circulation channel 36. Still other modifications are possible.

Controller 78 of thermal control unit 22 is contained within main body 30 of thermal control unit 22 and is in electrical communication with pump 34, heat exchanger 40, outlet temperature sensor 56, bypass valve 63, a patient temperature module 80, and a user interface 82. Controller 78 includes any and all electrical circuitry and components necessary to carry out the functions and algorithms described herein, as would be known to one of ordinary skill in the art. Generally speaking, controller 78 may include one or more microcontrollers, microprocessors, and/or other programmable electronics that are programmed to carry out the functions described herein. It will be understood that controller 78 may also include other electronic components that are programmed to carry out the functions described herein, or that support the microcontrollers, microprocessors, and/or other electronics. The other electronic components include, but are not limited to, one or more field programmable gate arrays, systems on a chip, volatile or nonvolatile memory, discrete circuitry, integrated circuits, application specific integrated circuits (ASICs) and/or other hardware, software, or firmware, as would be known to one of ordinary skill in the art. Such components can be physically configured in any suitable manner, such as by mounting them to one or more circuit boards, or arranging them in other manners, whether combined into a single unit or distributed across multiple units. Such components may be physically distributed in different positions in thermal control unit 22, or they may reside in a common location within thermal control unit 22. When physically distributed, the components may communicate using any suitable serial or parallel communication protocol, such as, but not limited to, CAN, LIN, Firewire, I-squared-C, RS-232, RS-465, universal serial bus (USB), etc.

User interface 82, which may be implemented as a control panel or in other manners, allows a user to operate thermal control unit 22. User interface 82 communicates with controller 78 and includes controls 84 enabling a user to turn control unit 22 on and off, select a mode of operation, select a target temperature for the fluid delivered to thermal pads 24, select a patient target temperature, input pone or more patient parameters, and control other aspects of thermal control unit 22. In some embodiments, user interface may include a pause/event control, a medication control, and/or an automatic temperature adjustment control that operate in accordance with the pause event control 66b, medication control 66c, and automatic temperature adjustment control 66d disclosed in commonly assigned U.S. patent application Ser. No. 62/577,772, filed on Oct. 27, 2017, by inventors Gregory Taylor et al. and entitled THERMAL SYSTEM WITH MEDICATION INTERACTION, the complete disclosure of which is incorporated herein by reference.

In some embodiments, user interface 82 includes a display 86. Display 86 may be implemented as a touch screen, or, in some embodiments, as a non-touch-sensitive display. When user interface 82 includes a touch screen display 86, one or more dedicated controls may also be included, such as one or more buttons, switches, dials, or other dedicated structures. When both a touch screen display and dedicated controls are included, any one or more of the functions carried out by a dedicated control may be replaced or supplemented with a touch screen control that is activated when touched by a user. Alternatively, one or more of the controls that are carried out via a touch screen display 86 can be replaced or supplemented with a dedicated control that carries out the same function when activated by a user. In some embodiments, display 86 is configured to include any of the display and control features disclosed in commonly assigned U.S. patent application Ser. No. 62/610,362 filed Dec. 26, 2017, by inventors Gregory S. Taylor, and entitled THERMAL SYSTEM WITH GRAPHICAL USER INTERFACE, the complete disclosure of which is incorporated herein by reference.

User interface 82 allows a user to select from different modes for controlling the patient's temperature. The different modes include, but are not limited to, a manual mode and an automatic mode, both of which may be used for cooling and heating the patient. In the manual mode, a user selects a target temperature for the fluid that circulates within thermal control unit 22 and that is delivered to thermal pads 24. Control unit 22 then makes adjustments to heat exchanger 40 in order to ensure that the temperature of the fluid exiting supply hoses 26a is at the user-selected temperature. In the automatic mode, the user selects a target patient temperature, rather than a target fluid temperature. After selecting the target patient temperature, controller 78 makes automatic adjustments to the temperature of the fluid in order to bring the patient's temperature to the desired patient target temperature. In this mode, the temperature of the circulating fluid may vary as necessary in order to bring about the target patient temperature.

In order to carry out the automatic mode, thermal control unit 22 utilizes patient temperature module 80. Patient temperature module 80 includes one or more patient temperature probe ports 88 (FIG. 2) that are adapted to receive one or more conventional patient temperature probes 90. The patient temperature probes 90 may be any suitable patient temperature probe that is able to sense the temperature of the patient at the location of the probe. In one embodiment, the patient temperature probes are conventional Y.S.I. 400 probes marketed by YSI Incorporated of Yellow Springs, Ohio, or probes that are YSI 400 compliant. In other embodiments, different types of probes may be used with thermal control unit 22. Regardless of the specific type of patient temperature probe used in thermal control system 20, each temperature probe 90 is connected to a patient temperature probe port 88 positioned on control unit 22. Patient temperature probe ports 88 are in electrical communication with controller 78 and provide current temperature readings of the patient's temperature.

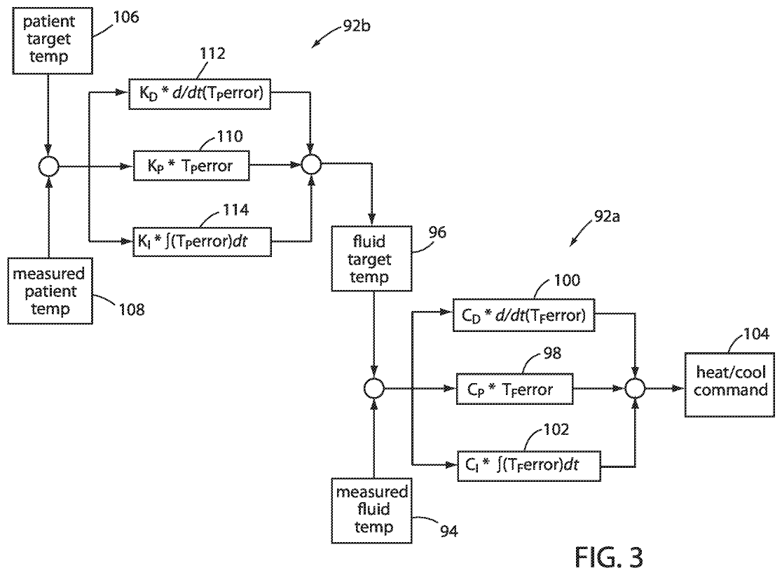

FIG. 3 illustrates a pair of feedback loops 92a and 92b that are used in at least one embodiment of thermal control unit 22. Feedback loop 92a is used by controller 78 when thermal control unit 22 is operating in the manual mode and feedback loops 92a and 92b are both used by controller 78 when thermal control unit 22 is operating in the automatic mode. Feedback loop 92a uses a measured fluid temperature 94 and a fluid target temperature 96 as inputs. Measured fluid temperature 94 comes from outlet temperature sensor 56. Fluid target temperature 96, when thermal control unit 22 is operating in the manual mode, comes from a user inputting a desired fluid temperature using controls 84 of user interface 82. When thermal control unit 22 is operating in the automatic mode, fluid target temperature 96 comes from the output of control loop 92b, as discussed more below.

Control loop 92a determines the difference between the fluid target temperature 96 and the measured fluid temperature 94 (T_{F}error) and uses the resulting error value as an input into a conventional Proportional, Integral, Derivative (PID) control loop. That is, controller 78 multiplies the fluid temperature error by a proportional constant (C_{P}) at step 98, determines the derivative of the fluid temperature error over time and multiplies it by a constant (C_{D}) at step 100, and determines the integral of the fluid temperature error over time and multiplies it by a constant (C_{I}) at step 102. The results of steps 98, 100, and 102 are summed together and converted to a heating/cooling command at step 104. The heating/cooling command is fed to heat exchanger 40 and tells heat exchanger 40 whether to heat and/or cool the circulating fluid and how much heating/cooling power to use.

Control loop 92b which, as noted, is used during the automatic mode, determines the difference between a patient target temperature 106 and a measured patient temperature 108. Patient target temperature 106 is input by a user of thermal control unit 22 using controls 84 of user interface 82. Measured patient temperature 108 comes from a patient temperature probe 90 coupled to one of patient temperature probe ports 88 (FIG. 2). Controller 78 determines the difference between the patient target temperature 106 and the measured patient temperature 108 (T_{P}error) and uses the resulting patient temperature error value as an input into a conventional PID control loop (FIG. 3). As part of the PID loop, controller 78 multiples the patient temperature error by a proportional constant (K_{P}) at step 110, multiplies a derivative of the patient temperature error over time by a derivative constant (K_{D}) at step 112, and multiplies an integral of the patient temperature error over time by an integral constant (K_{I}) at step 114. The results of steps 110, 112, and 114 are summed together and converted to a target fluid temperature value 96. The target fluid temperature value 96 is then fed to control loop 92a, which uses it to compute a fluid temperature error, as discussed above.

It will be understood by those skilled in the art that although FIG. 3 illustrates two PID control loops 92a and 92b, other types of control loops may be used. For example, loops 92a and/or 92b can be replaced by one or more PI loops, PD loops, and/or other types of control equations. Controller 78 implements loops 92a and/or 92b multiple times a second in at least one embodiment, although it will be understood that this rate may be varied widely. After controller 78 has output a heat/cool command at step 104 to heat exchanger 40, controller 78 takes another patient temperature reading 108 and/or another fluid temperature reading 94 and re-performs loops 92a and/or 92b. The specific loop(s) used, as noted previously, depends upon whether thermal control unit 22 is operating in the manual mode or automatic mode.

It will also be understood by those skilled in the art that the output of the control loop 92a may be limited such that the temperature of the fluid delivered to thermal pads 24 by thermal control unit 22 never strays outside of a predefined maximum and a predefined minimum. The predefined minimum temperature is a safety temperature below which controller 78 does not lower the temperature of the circulating fluid. In some embodiments, it may be set to about four degrees Celsius, although other temperatures may be selected. The predefined maximum temperature is also a safety temperature above which controller 78 does not heat the circulating fluid. The predetermined maximum temperature may be set to about forty degrees Celsius, although other values may be selected.

In some embodiments, controller 78 of thermal control unit 22 is programmed to determine a Q value. The Q value refers to the amount of heat being added to, or removed from, the patient via thermal pads 24. This value is calculated, in at least some embodiments, by determining the difference in temperature between the fluid delivered to the thermal pads 24 and the fluid returned from the thermal pads 24, and then multiplying this temperature difference by the flow rate (in mass per unit of time) and the specific heat capacity of the particular type of fluid (such as, but not limited to, water) being used with thermal control unit 22. The result is the amount of heat energy being delivered per unit of time via the thermal pads 24 (when being used to warm the patient) or the amount of heat energy being absorbed per unit of time via the thermal pads 24 (when being used to cool the patient). In some embodiments, the total quantify of heat delivered or absorbed during the thermal therapy session may be calculated and displayed. Further, in some embodiments, Q values may be calculated and displayed for each individual thermal pad 24, such as is disclosed in commonly assigned U.S. patent application Ser. No. 62/610,362, filed Dec. 26, 2017, by inventor Gregory S. Taylor, and entitled THERMAL SYSTEM WITH GRAPHICAL USER INTERFACE, the complete disclosure of which has been incorporated herein by reference.

As noted previously, user interface 82 is adapted to allow a user to input one or more patient parameters. These patient parameters include one or more non-temperature patient parameters, such as the patient's height, weight, body-mass index (BMI), body surface area (BSA), and/or other parameters. Controller 78 is adapted to use one or more of the entered non-temperature patient parameters to control heat exchanger 40 and deliver temperature-controlled fluid to thermal pads 24. In some embodiments, controller 78 selects one or more of the coefficients (C_{D}, C_{P}, C_{I}, K_{D}, K_{P}, and K_{I}) used in loops 92a and/or 92b based upon the entered non-temperature patient parameter. Thus, for example, in some embodiments, controller 78 is programmed to use a first set of coefficients when thermally treating a relatively small patient and to use a second and different set of coefficients when thermally treating a relatively large patient. In other embodiments, controller 78 is programmed to additionally or alternatively use the non-temperature patient parameter(s) in other manners when controlling heat exchanger 40, such as setting or changing limits on the temperature of the fluid delivered to the thermal pads 24, setting or changing limits on the rate of change of the temperature of the fluid delivered to the thermal pads 24, and/or in other manners.

In some embodiments, controller 78 is programmed in accordance with one or more of the algorithms disclosed in commonly assigned U.S. patent application Ser. No. 62/610,319 filed Dec. 26, 2017, by inventors Gregory S. Taylor et al. and entitled THERMAL SYSTEM WITH OVERSHOOT REDUCTION, the complete disclosure of which is incorporated herein by reference. When so programmed, controller 78 may be additionally programmed to utilize the non-temperature patient parameter entered via user interface 82 to select the value TA disclosed in that application. Additionally, or alternatively, when controller 78 is programmed to carry out any of the algorithms disclosed in the '319 application (THERMAL SYSTEM WITH OVERSHOOT REDUCTION) patent application, controller 78 may utilize the non-temperature patient parameter when evaluating the rate of change of the patient's temperature drop during step 112 of the algorithm 98 disclosed therein, and/or controller 78 may utilize the non-temperature patient parameter when determining switchover points B or D illustrated in FIG. 12 of that application. Still other manners of using the non-temperature patient parameter can be carried out with any of the algorithms disclosed in the '319 application, which, as noted, may be incorporated into the programming of controller 78 herein.

Additionally, as previously noted, controller 78 may be programmed in accordance with one or more of the algorithms disclosed in commonly assigned U.S. patent application Ser. No. 62/577,772, filed on Oct. 27, 2017, by inventors Gregory Taylor et al. and entitled THERMAL SYSTEM WITH MEDICATION INTERACTION, the complete disclosure of which is incorporated herein by reference. When so programmed, controller 78 carries out one or more of the algorithms disclosed therein using the non-temperature patient parameter entered via user interface 82. For example, in some embodiments, controller 78 uses the non-temperature patient parameter when predicting how a patient's temperature reacts during the administration of a medication, and/or predicting how a patient's temperature changes while a patient temperature probe is receiving erroneous readings due to a fluid flush taking place at the situs of the probe, and/or for other purposes.

In many embodiments, controller 78 is programmed to utilize the non-temperature patient parameter, such as BMI and/or weight, in order to control heat exchanger 40 in a manner that reduces overshoot. For example, patients having a higher BMI generally take longer to cool using thermal pads. This is because the heat removal via thermal pads 24 from the patient's periphery takes longer to affect the patient's core due to the larger amount of body mass between the patient's periphery and core. The opposite is also true. That is, adding heat to the patient via thermal pads 24 to the patient's periphery takes longer to affect the patient's core due to the larger amount of body mass between the patient's periphery and core. Controller 78 therefore controls heat exchanger 40 when treating a larger BMI patient in a manner that accounts for the greater delay between the time cold or warm fluid is applied to thermal pads 24 and the time the cold or warmth affects the patient's core. In some embodiments, controller 78 accounts for this greater delay by cooling (or heating, when appropriate) more aggressively when treating a higher-BMI patient than a lower BMI patient because the more aggressive cooling (or heating) is needed to change the patient's core temperature. Conversely, controller 78 is programmed to relax its cooling at a sooner point in time when the patient's core temperature nears target temperature 106 than it would for a lower BMI patient in order to reduce overshoot.

In some embodiments, controller 78 is programmed to use the BMI (or other non-temperature patient parameter, such as, but not limited to, patient weight) to calculate a standard heat capacitance value for the patient. Controller 78 then compares this heat capacitance with the amount of heat being provided or removed (Q value) from the patient via thermal pads 24. Based on this comparison, controller 78 adjusts the heat addition or heat removal via thermal pads 24 in order to match the standard heat capacitance. Controller 78 also uses the standard heat capacitance to predict where the patient's temperature trends are going in the future, and uses this information to adjust the control of heat exchanger 40 and the temperature of the fluid delivered to thermal pads 24 accordingly. Controller 78 is therefore programmed to control thermal control unit 22 in a manner that is partially custom-tailored to the individual patient undergoing thermal treatment. This provides improved performance over prior thermal control systems that provided the same thermal treatment to patients regardless of any individual non-temperature characteristics of those patients.

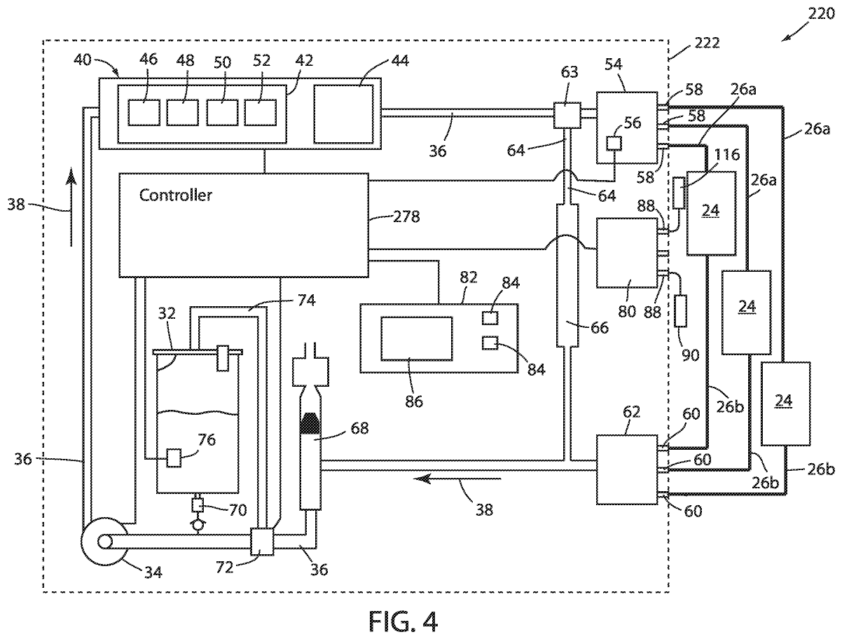

FIG. 4 illustrates a thermal control system 220 according to a second embodiment of the present disclosure. Those components of thermal control system 220 that are the same as, and operate in the same manner as, components of thermal control system 20 have been assigned the same reference number. Those components that are new have been assigned a new reference number, and those components that are modified have been assigned the same number increased by 200.

Thermal control system 220 differs from thermal control system 20 in that thermal control system 220 is adapted to provide thermal treatment utilizing patient feedback of not only the patient's core temperature, but also one or more patient peripheral temperatures. Thus, thermal control system 220 uses not only the patient's core temperature to control heat exchanger 40 and the temperature of fluid delivered to thermal pads 24, but also one or more peripheral temperatures of the patient. Thermal control unit 222 receives the patient peripheral temperature readings via patient temperature module 80. More specifically, patient temperature module 80 includes multiple patient temperature probe ports 88 and one of the ports 88 is coupled to a core temperature probe 90 and at least one of the other ports is coupled to a peripheral temperature probe 116. Core temperature probe 90 is placed at a location on the patient's body that measures a core temperature of the patient, such in the patient's rectum, esophagus, armpits, etc. Peripheral temperature probe 116 is placed at a selected location on the patient's body that measures a peripheral temperature of the patient, such as on the patient's skin, etc. In some embodiments, one or more peripheral temperature probes 116 are integrated into thermal pads 24. The readings from both temperature probes 116 and 90 are delivered by patient temperature module 80 to controller 278.

In some embodiments, thermal control unit 222 includes a designated port 88 for the core temperature probe 90 and one or other ports 88 that are designated for peripheral temperature readings from probe 116. In such embodiments, the user of thermal control system 220 plugs the core temperature probe 90 into the corresponding port 88 that is designated for core temperature readings and the peripheral temperature probe 116 into the corresponding port 88 that is designated for peripheral temperature readings. Controller 278 knows which port corresponds to which temperature reading and is therefore able to discern which temperature readings are core temperature readings and which temperature readings are peripheral temperature readings. In other embodiments, the user is free to plug in probes 90 and 116 to whichever ones of the ports 88 he or she likes. He or she instructs controller 278 which port 88 is coupled to peripheral probe 116 and which port 88 is coupled to the core probe 90 via user interface 82.

Regardless of whether ports 88 are hardware or software configured to accept core and peripheral temperature readings, thermal control unit 222 utilizes the core and peripheral temperature readings in its control of heat exchanger 40 and its delivery of temperature-controlled fluid to thermal pads 24. One manner in which controller 278 utilizes the core and peripheral temperature readings is by repetitively determining the difference between the two and monitoring changes in the difference. Controller 278 then makes changes to the manner in which it is controlling heat exchanger 40 if the difference between the two temperatures gets too large or too small.

In at least one embodiment, controller 278 is configured to determine the difference between the core and peripheral temperatures over time and use that information to determine what type of physiological response is occurring with the patient. If the difference is large and/or persists over time, controller 278 concludes that the patient is physiologically resisting the peripheral cooling/heating that is occurring via thermal pads 24. In such cases, controller 278 is programmed to be more aggressive in its heating or cooling in order to overcome the core temperature's resistance to homogenizing itself more closely with the patient's peripheral temperature. However, as the patient's core temperature approaches the target temperature 106, controller 278 is programmed to more quickly pull back on its thermal treatment based on second order temperature behavior of the patient (e.g. steps to prevent overshoot may need to occur sooner given the patient's greater lag between core temperatures matching the peripheral temperatures).

In contrast, if the difference between the patient's core and peripheral temperatures becomes (or remains) relatively small over time, the patient's core and peripheral temperature readings indicate a relatively homogenous temperature gradient within the patient, and such a homogenous gradient suggest the patient is putting up less physiological resistance to the thermal treatment. In such cases, controller 278 is programmed to be less aggressive in its heating/cooling because the temperature adjustments it applies to the patient's periphery via thermal pads 24 are more quickly and easily translated to the patient's core temperature.

In some embodiments, controller 278 is programmed to monitor the difference between the patient's core temperature and peripheral temperature and use that difference as a factor in determining whether or not to switch to a different set of one or more coefficients of loops 92a and/or 92b. In such embodiments, controller 278 may be programmed to use a first set of coefficients when the difference exceeds a threshold and a second set of coefficients when the difference is smaller than the threshold. Controller 278 may alternatively or additionally be programmed to switch between sets of coefficients based upon a rate of change of the difference between the core temperature and peripheral temperature. In any of these embodiments, one or more additional factors beyond the core-peripheral difference may also be considered by controller 278 when deciding whether to switch coefficients.

Still further, in some embodiments, controller 278 is programmed to monitor the difference between the patient's core temperature and peripheral temperature and take action to ensure that the difference does not exceed a predetermined maximum while the patient's temperature is being controlled. By limiting the difference between the peripheral and core temperatures, controller 278 helps to ensure that the thermal stress applied to the patient is limited. In other words, controller 278 is configured to ensure that a temperature gradient across the patient's body does not exceed a predetermined maximum, and this helps to reduce thermal stress on the patient. In some of these embodiments, controller 278 may take into account one or more factors about an individual patient when setting the predetermined limit. For example, patients with larger bodies may cause controller 278 to set a greater predetermined limit than patients with smaller bodies. Other factors besides weight, of course, can be used. Indeed, in some embodiments, user interface 82 is configured to allow a caregiver to set the predetermined limit for the temperature difference between the patient's core and peripheral temperatures.

In some embodiments, controller 278 is modified to infer a patient peripheral temperature based upon the amount of heat being transferred to, or removed from, the patient. In such embodiments, controller 278 determines the Q value and uses the Q value to infer a peripheral temperature. In such embodiments, controller 278 may also use the patient's weight and/or the patient's core temperature in order to infer the patient's peripheral temperature. Controller 278 infers patient peripheral temperatures by repeatedly calculating the amount of heat transferred to or from the patient, looking at the temperature of the fluid returning from thermal pads 24, and estimating based on these factors and (in some cases) the patient's weight, BMI, core temperature, and/or rate of change of core temperature, the peripheral temperature of the patient. The inference may be based on empirical data previously gathered for patients of different sizes and/or based on one or more conventional models of human thermal physiology. Regardless of the specific manner used to infer the temperature, controller 278 uses this inferred patient peripheral temperature to carry out any of the previously mentioned functions that controller 278 implements using actual readings from peripheral temperature probe 116. By inferring a patient peripheral temperature, a user of thermal control system 220 does not have to worry about positioning a peripheral temperature sensor 116 at a specific place or monitoring its position and function during the course of a thermal treatment session.

In some modified embodiments, peripheral temperature probe 116 may be integrated into one or more of the thermal pads 24. Although other designs may be used, some suitable examples of thermal pads incorporating temperature sensors that may be used for detecting a peripheral temperature reading are found in commonly assigned U.S. patent application Ser. No. 62/425,813 filed Nov. 23, 2016, by inventors Gregory Taylor et al. and entitled THERMAL SYSTEM, as well as commonly assigned U.S. patent application Ser. No. 15/675,066 filed Aug. 11, 2017, by inventor James K. Galer and entitled THERMAL SYSTEM, the complete disclosures of both of which are hereby incorporated by reference in their entirety herein.

Regardless of whether controller 278 receives direct patient peripheral temperature readings or infers such temperatures, controller 278 may be programmed to use the difference between the patient's core and peripheral temperatures in other manners besides switching coefficients. For example, in another modified embodiment, controller 278 is programmed to use the difference between the core and peripheral temperatures to set one or more limits on the integral term of either or both of the control loops 92a and 92b. Still other manners of using the difference between the core and peripheral temperatures are possible.

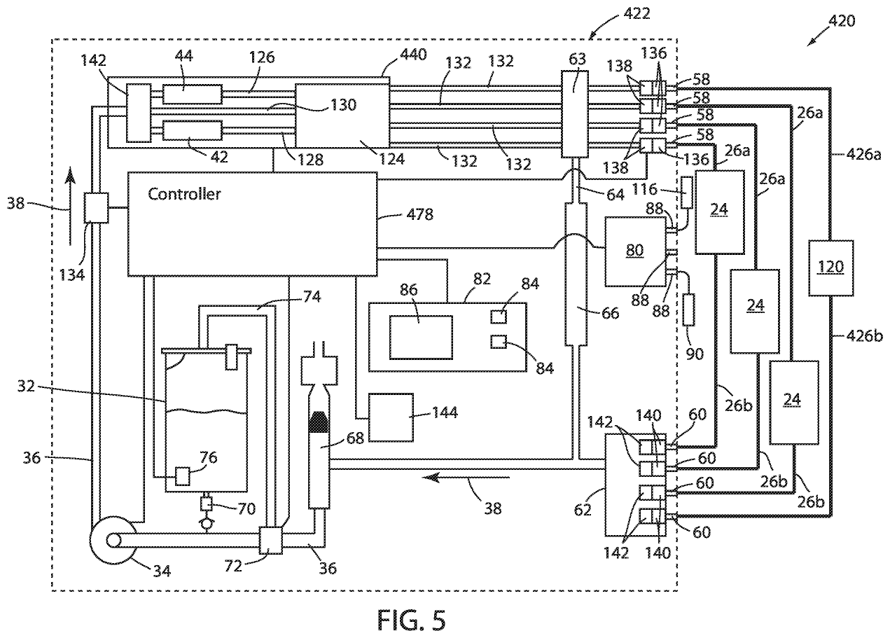

FIG. 5 illustrates a thermal control system 420 according to a second embodiment of the present disclosure. Those components of thermal control system 420 that are the same as, and operate in the same manner as, components of thermal control systems 20 and/or 220 have been assigned the same reference number. Those components that are new have been assigned a new reference number, and those components that are modified have been assigned the same number increased by 200 or 400.

Thermal control system 420 differs from thermal control system 220 in that thermal control system 220 is adapted to individually and separately control the temperature of the fluid that is exiting from each of the fluid outlet ports 58. That is, thermal control unit 422 is adapted to supply fluid, as appropriate, to outlet ports 58 with up to four different temperatures. Thus, thermal control unit 422 is adapted to supply fluid of different temperatures to each of the three thermal pads 24, as well as auxiliary fluid of yet another potentially different temperature, to an auxiliary thermal therapy device 120. Auxiliary thermal therapy device 120 is fluidly coupled to thermal control unit 422 by way of an additional fluid supply hose 426a and an additional fluid return hose 426b. As will be discussed in more detail below, the auxiliary fluid supplied to auxiliary thermal therapy device 120 may be the same fluid as that supplied to thermal pads 24, or it may be a different fluid.

Heat exchanger 440 of thermal control unit 422 is able to deliver fluid with independently controlled temperatures by using a set of inlet valves 122 and a set of outlet valves 124. Inlet valves 122 divide the incoming fluid into one or more of three possible paths through heat exchanger 440. These three paths include a heating path 126, a cooling path 128, and a neutral path 130. Heating path 126 passes through a heater 44; cooling path 128 passes through a chiller 42; and neutral path 130 does not pass through either a heater or a chiller. Each path 126, 128, and 130 feeds into outlet valves 124 which, like inlet valves 122, are under the control of controller 478. Controller 478 controls the outlet valves 124 such that the heated fluid from path 126, the cooled fluid from path 128, and the unchanged fluid from path 130 are mixed in the proper proportions to deliver fluid at the desired temperature to four outlet channels 132. Each outlet channel 132 is fluidly coupled to a corresponding outlet port 58.

Controller 478 controls the inlet and outlet valves 122 and 124 based on the incoming fluid temperature, which is sensed by temperature sensor 134. Controller 478 uses the output from temperature sensor 134, along with the target temperature for each fluid outlet channel 132 to determine how much fluid to direct along each of the paths 126, 128, and 130 and how to mix the fluid from each path, via outlet valves 124, such that the fluid delivered to each outlet channel 132 matches the target temperature for that outlet.

By delivering fluid with independently controlled temperatures to each of the outlet ports 58, thermal control unit 422 is able to provide different levels of heating and/or cooling to the individual thermal pads 24 applied to a patient 28. In this manner, for example, fluid of a first temperature might be delivered to the thermal pad 24 in contact with the patient's torso, while fluid of a second temperature might be delivered to the thermal pads 24 in contact with the patient's thighs. Alternatively, fluid of different temperatures might be delivered to all three thermal pads 24. Still other combinations of temperatures for the thermal pads 24 are also possible.

Thermal control unit 422 also differs from thermal control units 22 and 222 in that it includes a plurality of flow control valves or restrictors 136. Each restrictor 136 is positioned in the fluid path of one of the four outlet ports 58. Restrictors 136 are under the control of controller 478 and allow controller 478 to control the amount of fluid that is output from outlet ports 58. Controller 478 is thereby able to independently control both the temperature of the fluid delivered to each outlet port 58 and the amount of fluid delivered to each outlet port 58.

In the illustrated embodiment of FIG. 5, thermal control unit 422 also includes an outlet temperature sensor 138 for each of the outlet ports 58. These temperature sensors 138 may be included in order to allow controller 478 to use positive feedback when mixing and controlling the fluid flow inside of heat exchanger 440. These may also be included in order for controller 478 to calculate the Q value (or heat quantity) that is delivered to each thermal pad 24 and auxiliary thermal therapy device 120, or absorbed by each thermal pad 24 and auxiliary thermal therapy device 120, as will be discussed in greater detail below.

Thermal control unit 422 also differs from thermal control units 22 and 222 in that it includes individual inlet temperature sensors 140 and individual flow meters 142 positioned inside, or in line with, inlet manifold 62. Each inlet temperature sensor 140 measure the temperature of the fluid returning from a corresponding thermal pad 24 and reports the temperature to controller 478. Each flow meter 142 measures the flow rate of the fluid returning from a corresponding thermal pad 24 or auxiliary thermal therapy device 120 and reports to the measured flow rate to controller 478. Controller 478 uses the individual temperatures and flow rates for purposes discussed in more detail below, such as the calculation of Q values for each thermal pad 24 and auxiliary thermal therapy device 120, and/or for feedback purposes (e.g. flow meters 142 may be used as closed loop feedback for controlling restrictors 136).

Thermal control unit 422 also differs from thermal control units 22 and 222 in that it includes one or more transceivers 144 for communicating with one or more external devices. In some embodiments, transceiver 144 is a wireless transceiver, such as, but not limited to, a Bluetooth transceiver (IEEE 802.15.1), a ZigBee transceiver (IEEE 802.15.4), or a WiFi transceivers (IEEE 802.11). In other embodiments, transceiver 144 is adapted to communicate using a wired connection and may utilize a USB port, a DB-25 connector, a DE-9 connector, an 8P8C connector, or the like. When communicating using a wired connection, transceiver 144 may be configured to communicate using a variety of different communication protocols, including, but not limited to, Controller Area Network (CAN), Ethernet, TCP/IP, I-Squared-C, etc. In some embodiments, transceiver 144 and controller 478 are configured to share and/or receive information from other thermal devices in any of the manners disclosed in commonly assigned U.S. patent application Ser. No. 15/616,574 filed Jun. 7, 2017, by inventors Gregory S. Taylor et al. and entitled THERMAL CONTROL SYSTEM, the complete disclosure of which is incorporated herein by reference. Other types of communication, including those discussed in more detail below, are also possible.

Controller 478 of thermal control system 420 differs from controllers 78 and 278 in that controller 478 is adapted to implement a thermal therapy session using both thermal pads 24 and one or more auxiliary thermal therapy devices 120. Controller 478 is therefore configured to control not only the temperature (and in some embodiments, the flow rate) of the fluid delivered to thermal pads 24, but also the temperature (and in some embodiment, the flow rate) of the fluid delivered to auxiliary thermal therapy device 120. Thermal control unit 422 therefore controls the temperature of the patient's body using multiple types of thermal therapy devices.

In at least one embodiment, controller 478 is adapted to control an auxiliary thermal therapy device 120 that is in direct thermal communication with the patient's core temperature, and therefore able to more directly affect the patient's core temperature than the thermal pads 24. In these embodiments, controller 478 uses thermal pads 24 to deliver cold or warm fluid to thermal pads 24 in order to directly affect the patient's peripheral temperature, while simultaneously delivering cold or warm fluid to the auxiliary thermal therapy device 120 in order to directly affect the patient's core temperature.

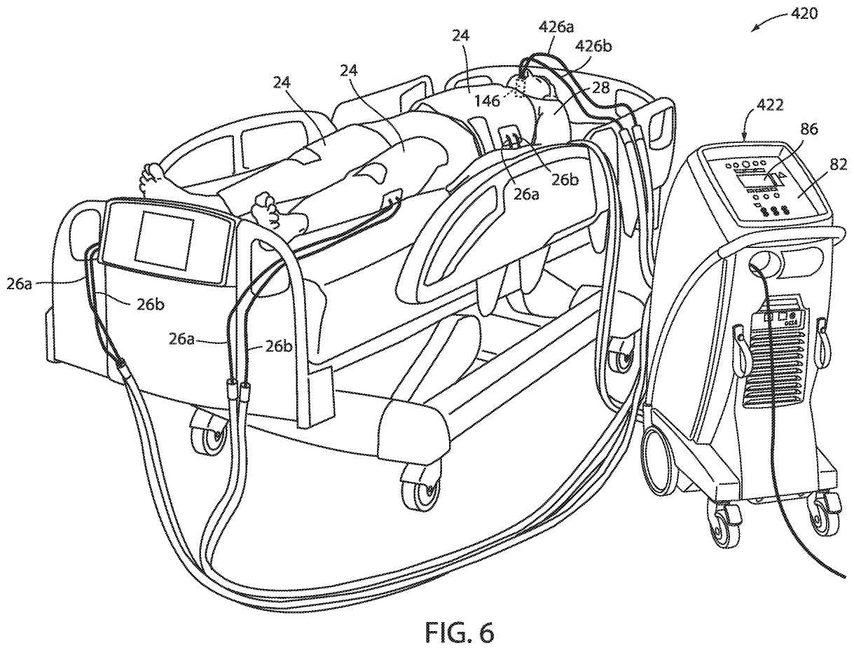

In at least one embodiment, auxiliary thermal therapy device 120 is an esophageal heat transfer device 146 that is inserted into the esophagus of a patient undergoing thermal treatment, such as shown in FIG. 6. One such suitable esophageal heat transfer device 146 is the ensoETM available from Attune Medical of Chicago, Ill.. The ensoETM is inserted into a patient's esophagus and comes into contact with the esophageal mucosa, allowing blood passing through the patient's blood vessels to be cooled or warmed by the temperature-controlled fluid circulating through the ensoETM. Still other types of esophageal heat transfer devices 146 may be used. Regardless of the specific type, thermal control unit 422 displays the temperature of the fluid delivered to the esophageal thermal transfer device and/or other information about the device. Thermal control unit 422 also determines the temperature of the fluid returning from esophageal heat transfer device 146 and, in at least some embodiments, determines a Q value for the heat transfer between the patient 28 and the device 146.

Controller 478 is adapted to receive both peripheral and core temperature readings, such as from peripheral temperature probe 116 and core temperature probe 90. Controller 478 is adapted to utilize these two temperatures in any of the manners discussed above with respect to controller 278. Thus, in some embodiments, controller 478 repetitively calculates and monitors the difference between the patient's core and peripheral temperatures and uses the temperature difference to control the temperature of the fluid delivered to the thermal pads 24. In contrast to controller 278, however, controller 478 uses the temperature difference to control the temperature of fluid delivered to auxiliary thermal therapy device 120 as well.

In most embodiments, controller 478 is configured to set the temperature of the fluid delivered to auxiliary thermal therapy device 120 to a temperature that is between the patient target temperature 106 and the fluid temperature 94 of the fluid delivered to the thermal pads 24. This is because the auxiliary thermal therapy device 120 is generally used to directly transfer heat to and/or from the patient's core. As such, it does not need to be as cold as the fluid delivered to thermal pads 24 when cooling the patient, nor does it need to be as warm as the fluid delivered to the thermal pads when warming the patient. Controller 478 therefore sets a target temperature for the fluid delivered to auxiliary thermal therapy device 120 that is different from the target temperature for the fluid delivered to one or more of the thermal pads 24.

In some instances, as discussed more below, the fluid delivered to auxiliary thermal therapy device 120 may be colder than the fluid in thermal pads 24 when cooling the patient and warmer than the fluid in thermal pads 24 when warming the patient. In still other embodiments, controller 478 may be configured to implement one or more separate control loop(s) 92a and/or 92b for controlling the temperature of the fluid delivered to auxiliary thermal therapy device 120 that are different from the control loop(s) 92a and/or 92b used to control the temperature of the fluid delivered to thermal pads 24. Finally, in some embodiments, auxiliary thermal therapy device 120 may be used with a thermal control unit that does not independently set the fluid temperatures of the fluid delivered to device 120 and thermal pads 24.

Although thermal control unit 422 has been described herein as delivering the same fluid to auxiliary thermal therapy device 120 and thermal pads 24, it will be understood that thermal control unit 422 can be modified to deliver a different auxiliary fluid to auxiliary thermal therapy device 120. In such modified embodiments, thermal control unit 422 includes a separate set of fluid channels for the auxiliary fluid that keep the auxiliary fluid separate from the fluid delivered to thermal pads. A separate heater/chiller may also be included. The desired temperature for the auxiliary fluid is determined in any of the same manners discussed above for determining the fluid delivered to thermal pads 24. Alternatively, the temperature of the auxiliary fluid may be controlled using one or more control loops 92a and/or 92b that are separate from the control loops 92a and/or 92b used to control the temperature of the fluid delivered to thermal pads 24.

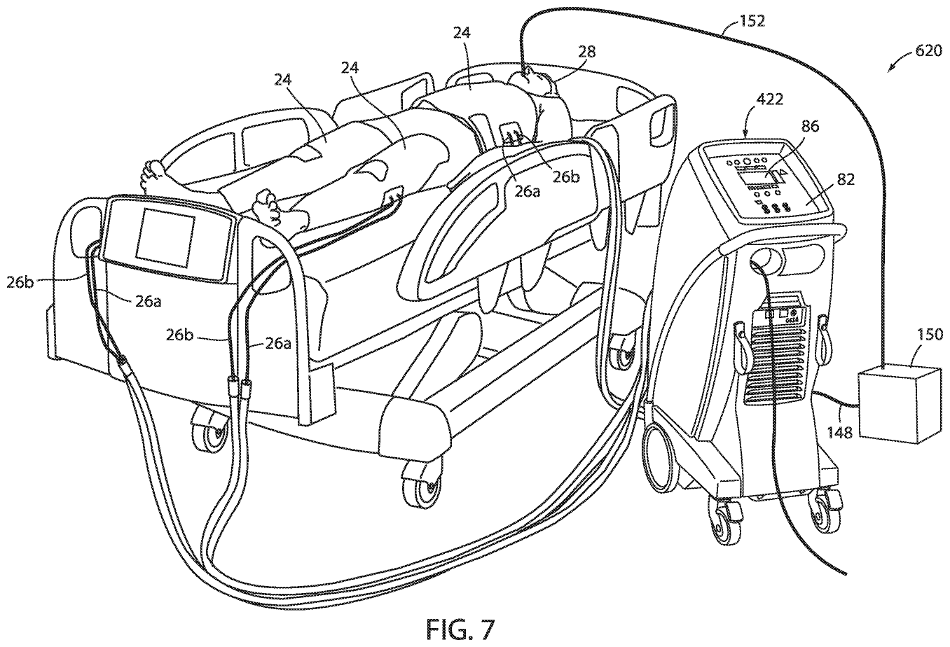

FIG. 7 illustrates a thermal control system 620 according to another embodiment of the present disclosure. Those components of thermal control system 620 that are the same as, and operate in the same manner as, components of thermal control systems 20, 220, and/or 420 have been assigned the same reference number. Those components that are new have been assigned a new reference number, and those components that are modified have been assigned the same number increased by 200, 400, or 600.

Thermal control system 620 utilizes thermal control unit 422 of FIGS. 5 & 6 along with a different thermal therapy device 120 from the one used in thermal control system 420. Specifically, thermal control system 620 utilizes an auxiliary thermal therapy device 120 that is implemented as an air controller 150 rather than an esophageal heat transfer device 146. Air controller 150 controls the temperature of the air breathed in by patient 28, and in some embodiments, the humidity and/or pressure of the air delivered to the patient. Air controller 150, unlike esophageal heat transfer device 146, couples to thermal control unit 422 via transceiver 144 rather than via hoses 426a and 426b. That is, in the embodiment illustrated in FIG. 7, thermal control unit 422 does not deliver temperature-controlled fluid to air controller 150. Instead, air controller 150 includes its own heating/cooling structures that heat and cool the air according to instructions received from thermal control unit 422. Thermal control unit 422 communicates these instructions to air controller 150 via transceiver 144. In the embodiment shown in FIG. 7, transceiver 144 is a wired transceiver that allows communication between thermal control unit 422 and air controller 150 via a cable 148. In an alternative embodiment, transceiver 144 is a wireless transceiver and communicates with air controller 150 wirelessly.

Air controller 150 is coupled to an air hose 152 that is fed to the patient's nose and/or mouth. Although not shown in FIG. 7, air hose 152 may be coupled to a breathing mask that covers the patient's nose and/or mouth, or it may be coupled to another type of device that holds the air hose 152 in place so that the temperature-controlled air delivered from air controller 150 to the patient via air hose 152 is available for the patient to breathe in. The breathing in of the temperature-controlled air by the patient helps to heat or cool the patient, depending upon the temperature of air. Controller 478 of thermal control unit 422 controls the temperature of the air in any of the same manners controller 78 controls the temperature of the fluid delivered to thermal therapy pads 24. Such control, however, may be modified such that, instead of controlling the air temperature directly, controller 478 sends instructions for controlling the air temperature to air controller 150 which then implements the instructions.

In some modified embodiments, thermal control unit 422 of thermal control system 620 may be modified to include air temperature control structures within itself. In such modified embodiments, thermal control unit 422 directly controls the temperature of the air and supplies it to patient 28, thereby avoiding the need for a separate air controller 150. Thus, thermal control unit 422 may be modified to essentially integrate air controller 150 within itself such that air hose 152 couples to thermal control unit 422 instead of a separate air controller 150. In such modified embodiments, thermal control unit 422 may include a heat exchanger separate from heat exchanger 440 for controlling the temperature of the air, or it may utilize the same heat exchanger 440 for controlling both the fluid delivered to thermal pads 24 and the auxiliary fluid (air) delivered to hose 152. One example of a thermal control unit that delivers both temperature-controlled liquid and temperature-controlled air to a patient for thermal treatment of the patient is disclosed in commonly assigned U.S. patent application Ser. No. 15/675,061 filed Aug. 11, 2017, by inventors James Galer et al. and entitled THERMAL THERAPY DEVICES, the complete disclosure of which is incorporated herein by reference. Other types of thermal control units that supply temperature-controlled air and liquid may also be used.

In some embodiments, air controller 150 is a conventional ventilator, or other breathing assistance device, that includes temperature, humidity, and/or pressure controls. In such embodiments, controller 478 sends instructions for controlling the air temperature, humidity, and/or pressure to air controller 150 which then implements the instructions. The air temperature, humidity, and/or pressure are selected in order to complement the control of the patient's temperature being carried out by thermal control unit 422 and thermal pads 24. Thus, when controller 478 is cooling the patient via thermal pads 24, controller 478 also controls the air temperature, humidity, and/or pressure of the air in order to assist in this cooling. Similarly, when controller 478 is warming the patient via thermal pads 24, controller 478 also controls the air temperature, humidity, and/or pressure of the air to assist in this warming.

In at least one embodiment, controller 478 is configured to do one or both of the following at certain times during the thermal treatment of a patient: (1) warm the patient via air controller 150 while thermal pads 24 are cooling the patient, and (2) cool the patient via air controller 150 while the thermal pads 24 are warming the patient in some instances. Such instances may occur when the patient is approaching the patient target temperature 106, and controller 478 is seeking to prevent overshoot of the patient target temperature 106. Thus, for example, because thermal pads 24 do not immediately affect the core temperature of the patient (due to their being positioned externally on the patient), controller 478 may alter the temperature of the fluid supplied to thermal pads 24 as the patient approaches target temperature 106 and do so prior to altering the temperature, pressure and/or humidity of the air supplied by air controller 150.

In the case of cooling the patient, when controller 478 detects that the patients' temperature is close to the target temperature (and will likely reach the target temperature without further cooling using thermal pads 24), controller 478 may stop cooling the patient using thermal pads 24 while continuing to cool the patient using air controller 150. While the cooling using thermal pads 24 is stopped, controller 478 may stop supplying fluid to thermal pads 24 and/or may start heating the fluid supplied to thermal pads 24 so that heat exchanger 440 has time to overcome the thermal inertia of the circulating fluid and warm it to a temperature that—when delivered to the patient at or near the moment the patient reaches the target temperature—assists in reducing or preventing temperature overshoot. Controller 478 may perform the opposite when warming the patient. That is, controller 478 may stop warming the patient using thermal pads 24 prior to the patient reaching target temperature 106 but continue to use air controller 150 to warm the patient. After stopping the warming via thermal pads 24, controller 478 may begin cooling the circulating fluid internally within thermal control unit 422 and deliver the cooled fluid to the patient at or near the moment the patient reaches the target temperature, thereby helping to reduce or eliminate overshoot. Further details of several manners in which controller 478 may control the temperature of the circulating fluid in order to reduce or eliminated patient temperature overshoot are disclosed in commonly assigned U.S. patent application Ser. No. 62/610,319 filed Dec. 26, 2017, by inventors Gregory Taylor et al. and entitled THERMAL SYSTEM WITH OVERSHOOT REDUCTION, the complete disclosure of which is incorporated herein by reference.

In some embodiments, air controller 150 includes one or more sensors for measuring not only the temperature of the air delivered to the patient, but also the temperature of the air exhaled by the patient. In such embodiments, air controller 150 may also include sensors for measuring the humidity and pressure of the inhaled air, as well as the humidity and pressure of the exhaled air. Using information supplied by these sensors, controller 478 is configured to calculate how much heat is being added to or removed from the patient via the patient's breathing. In such embodiments, controller 478 may utilize the following two equations for calculating this heat quantity:
Q_{lung(conv)}=\dot{m}(C_{P})(Tₑₓₕₐₗₑ−Tᵢₙₕₐₗₑ) and
Q_{lung(latent)}=\dot{m}(h_{fg})(Wₑₓₕₐₗₑ−Wᵢₙₕₐₗₑ)
where Q_{lung(conv)} is the heat transferred due to convection;
Q_{lung(latent)} is the heat transferred due to evaporation;
\dot{m} is the rate of air intake to the lungs (kg/s)
C_{P} is the specific heat of air;
Tₑₓₕₐₗₑ and Tᵢₙₕₐₗₑ are the temperatures of the exhaled and inhaled air, respectively;
h_{fg} is the enthalpy of vaporization of water; and
Wₑₓₕₐₗₑ and Wᵢₙₕₐₗₑ are the humidity ratios of the exhaled and inhaled air (mass of moisture per unit mass of dry air).

In addition to utilizing this heat transfer information to determine how to control the temperature, pressure, and/or humidity of the air delivered to the patient via air controller 150 (and/or the temperature of the fluid delivered to one or more thermal pads 24), controller 478 is configured in some embodiments to display calculated convection and evaporation heat transfer values, or a total heat transfer value (convection value plus the evaporation value). In some embodiments, this information is displayed in conjunction with heat transfer values calculated for the thermal pads 24, such as is disclosed in commonly assigned U.S. patent application Ser. No. 62/610,362 filed Dec. 26, 2017, by inventor Gregory S. Taylor and entitled THERMAL SYSTEM WITH GRAPHICAL USER INTERFACE, the complete disclosure of which has been incorporated herein by reference.

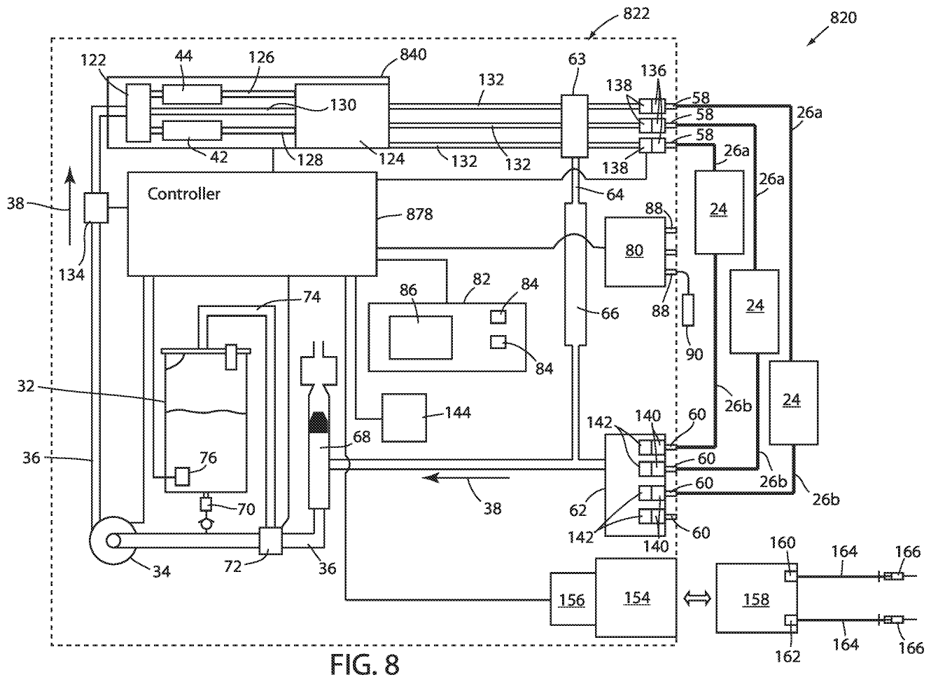

FIG. 8 illustrates a thermal control system 820 according to yet another embodiment of the present disclosure. Those components of thermal control system 820 that are the same as, and operate in the same manner as, components of thermal control systems 20, 220, 420, and/or 620 have been assigned the same reference number. Those components that are new have been assigned a new reference number, and those components that are modified have been assigned the same number increased by 200, 400, 600, or 800.

Thermal control system 820 differs from the other thermal control systems described herein in that thermal control system 820 is adapted to simultaneously control the temperature of a different auxiliary fluid, in addition to controlling the temperature of fluid delivered to thermal pads 24. The different auxiliary fluid is specifically the patient's blood. Thermal control system 820 includes a thermal control unit 822 having a cartridge receptacle 154 adapted to receive a cartridge 158 through which the patient's blood flows (FIG. 8). Cartridge receptacle 154 is positioned adjacent to a cartridge interface 156. Cartridge interface 156 may include a heat exchanger adapted to change the temperature of the blood flowing through the adjacent cartridge, and/or it may include an interface for communicating with a heat exchanger contained within the cartridge itself. In either case, cartridge interface 156 is in communication with controller 878 and operates under the control of controller 878.

Cartridge 158 includes an outlet 160 and an inlet 162 that are each fluidly coupled to a hose 164. Each hose 164 is coupled to an IV needle 166. In the illustrated embodiment, the patient's blood is directed to cartridge 158 by inserting a first one of the IV needles 166 into a vein of the patient and inserting the second one of the IV needles 166 into another vein of the patient. The veins to which the IV needles 166 are coupled are peripheral veins, in at least one embodiment. For example, in at least one embodiment, a first one of the IV needles 166 is inserted into the median cubital vein of a first one of the patient's arms and a second one of the IV needles 166 is inserted into the medial cubital vein of the second one of the patient's arms. A pump is included within cartridge 158 in order to draw blood from the first one of the veins and deliver it to the other one of the veins after passing through cartridge 158 and being thermally treated within cartridge 158. In other embodiments, needles 166 may be coupled to other ones of the patient's veins, and in some embodiments, needles 166 may be coupled to the same vein of the patient's.

In many of the embodiments, cartridge 158 is constructed such that it can be used in conjunction with both thermal control unit 822 and a more portable thermal control unit that is easily transportable by emergency personnel who respond to patient emergencies. Thus, in some embodiments, cartridge 158 is designed to be used with any of the portable thermal control units disclosed in commonly assigned U.S. patent application Ser. No. 15/460,988 filed Mar. 16, 2017, by inventor Gregory S. Taylor and entitled MOBILE THERMAL SYSTEM , the complete disclosure of which is incorporated herein by reference. Other types of portable thermal control units may also accept cartridge 158 and control the temperature of the patient's blood flowing therethrough while the portable thermal control unit is in the field with the patient.

By using a cartridge 158 that is compatible with both thermal control unit 822 and a portable thermal control unit, an emergency worker can utilize the portable thermal control unit while attending to a patient in the field and then have that person's thermal treatment easily transferred to thermal control unit 822 when the patient is brought to a hospital or other medical facility. The cartridge 158 is simply removed from the portable thermal control unit and inserted into the cartridge receptacle 154 of thermal control unit 822. To the extent any data was gathered by the portable thermal control unit, this is transferable to thermal control unit 822 in any of the manners disclosed in commonly assigned U.S., patent application Ser. No. 15/616,574 filed Jun. 7, 2017, by inventors Gregory S. Taylor et al. and entitled THERMAL CONTROL SYSTEM , the complete disclosure of which is incorporated herein by reference.

After the cartridge 158 is transferred to thermal control unit 822, the thermal treatment initiated by the portable thermal control unit may be continued using thermal control unit 822, or a caregiver can make any desired changes to the thermal treatment using user interface 82 of thermal control unit 822. In addition, the caregiver can add, if desired, one or more thermal pads 24 to the thermal treatment of the patient by coupling the pads 24 to the outlet and inlet ports 58 and 60 of thermal control unit 822 (the portable thermal control unit does not necessarily include the structures required to control the temperature of any thermal pads for thermally treating the patient). The addition of the thermal pads 24 to the thermal treatment provided by controlling the temperature of the patient's blood allows thermal control unit 822 to more quickly adjust the temperature of the patient.

Another advantage of cartridge 158 and its associated IV needles 166 is that the needles 166 are designed to be inserted into a patient in the same manner as conventional IV needles, which is a technique that emergency personnel are capable of doing. Thus, if an emergency caregiver decides that thermal treatment would be useful for a patient while the patient is out in the field, the caregiver does not need any additional training to insert the needles 166 into the patient's vein(s). This is unlike some conventional blood temperature control devices that require a catheter to be skillfully inserted into the patient's core through a peripheral location, which is a technique usually only performed in hospitals with specially trained doctors. Cartridge 158 and its associated IV needles therefore allow thermal treatment to be started by conventionally trained emergency personnel. Further, because the thermal therapy associated with IV needles 166 when it is applied in the field does not involve the use of any thermal pads 24, this field-initiated thermal therapy does not present any interference issues with respect to the emergency personnel accessing the patient's torso and/or legs. The emergency personnel are therefore able to perform CPR and/or provide other treatment or attention to whatever portion of the patient's body the situation requires without interference from thermal pads 24 and/or needles 166 and cartridge 158.

In some embodiments, hoses 164 and/or cartridge 158 are modified from the embodiments shown in the drawings to include a junction for connecting a conventional IV bag containing fluids and/or medication. In this manner, blood from the patient is withdrawn via one of the IV needles 166, the blood passes through a hose 164 to cartridge 158, and during passage through the hose 164 and/or through cartridge 158, fluid and/or medication is added to the patient's blood. The fluid and/or medication is then delivered to the patient through the other hose 164 and needle 166. This modification allows thermal treatment and medication/fluid treatment to be combined into a single unit. Further, because emergency personnel often install an IV into a patient while in the field, the installation of one additional needle 166 into the patient involves very little extra work. When modified in this manner, cartridge 158 and IV needles 166 allow the emergency technician to decide whether to apply only medication/fluid treatment to the patient, or to provide both medication/fluid treatment and thermal treatment to the patient. In other words, when a conventional IV bag is coupled to a junction of one of hoses 164 and/or cartridge 158, the emergency technician can utilize cartridge 158 and its associated thermal control unit to merely pump the fluid, as appropriate, to the patient without heating or cooling the blood. Alternatively, the emergency technician can instruct the thermal control unit to heat or cool the blood. In some further modified embodiments, cartridge 158 is used with a modified needle having two flow passages so that a single needle can be used to draw and return blood from a single vein of the patient, thereby obviating the need to insert multiple needles into the patient. Still other modifications can be made.

Once a patient undergoing thermal treatment is brought from the field into a hospital, or other healthcare facility, the portable thermal control unit utilized with cartridge 158 and IV needles 166 may continue to be used by the healthcare providers to treat the patient, or the healthcare providers can remove cartridge 158 from the portable thermal control unit and insert it into thermal control unit 822 (FIG. 8). Once inside thermal control unit 822, controller 878 takes over the control of thermal treatment of the blood (and/or other fluid/medication) flowing through cartridge 158. Thermal control unit 822 controls the temperature of the blood in any of the same manners discussed above with respect to thermal control units 422, 222, and 22. The only difference is that the fluid whose temperature is controlled by thermal control unit 822 is blood, instead of air, water, or another type of liquid used with these thermal control units. In some embodiments, controller 878 controls the temperature of the patient's blood in cartridge 158 in the same manner as thermal control unit 422 controls the temperature of the fluid supplied to esophageal heat transfer device 146, and in such cases controller 878 controls the heating and cooling of cartridge 158 as if it were an auxiliary thermal therapy device 120 and the blood flowing therein were an auxiliary fluid.

After a patient is brought from the field into a hospital or medical facility and cartridge 158 is transferred to cartridge receptacle 154, the healthcare personnel have the option of applying thermal pads 24 to the patient in order to expedite the heating or cooling of the patient, or omitting the thermal pads 24 and leaving the thermal treatment of the patient to be performed solely by heating/cooling the patient's blood flowing through cartridge 158. If applying thermal pads 24 to the patient, the thermal pads 24 are coupled to ports 58 and 60 of thermal control unit 822 and controller 878 controls heat exchanger 840 in a manner that brings the patient's core temperature to a target patient temperature.

The physical construction of cartridge 158 and cartridge receptacle 154 may vary widely. In some embodiments, cartridge 158 and receptacle 154 are constructed in accordance with any of the cartridge and receptacle designs disclosed in commonly assigned U.S. patent application Ser. No. 62/451,121 filed Jan. 27, 2017, by inventors Martin Stryker et al. and entitled THERMAL CONTROL SYSTEM WITH FLUID CARTRIDGES, the complete disclosure of which is incorporated herein by reference. When constructed in accordance with any of the designs disclosed in the '121 application, cartridge interface 156 may include the motor 46 and/or heat exchanger portions 48a, b disclosed therein. Alternatively, other cartridge designs may be used, including ones in which the heat exchanger and/or pump are integrated into the cartridge.

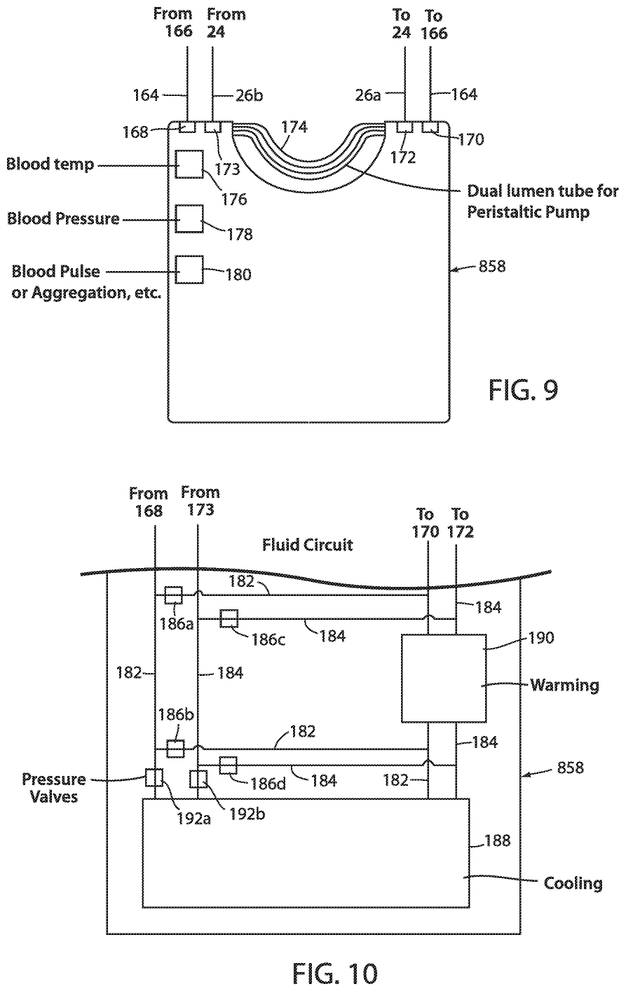

FIGS. 9 & 10 illustrate another embodiment of a cartridge 858 according to another aspect of the present disclosure. Cartridge 858 differs from cartridge 158 in that cartridge 858 includes its own heat exchangers. Cartridge 858 also differs from cartridge 158 in that cartridge 858 is adapted to circulate both blood and a non-blood fluid (e.g. water) therethrough. More specifically, cartridge 858 includes a blood inlet port 168, a blood outlet port 170, a fluid outlet port 172, and a fluid inlet port 173. Blood inlet port 168 couples to a hose 164 having a first IV needle 166 coupled thereto. Blood outlet port 170 couples to another hose 164 having a second IV needle 166 coupled thereto. Fluid outlet port 172 couples to a supply hose 26a that delivers fluid to a thermal pad 24 and fluid inlet port 173 couples to a return hose 26b that returns fluid from the thermal pad 24 to the thermal control unit 822.

As shown more clearly in FIG. 9, cartridge 858 includes a dual lumen tube 174. Dual lumen tube 174 allows both the blood from hoses 164 and the non-blood liquid from hose 26a to flow therethrough. When flowing therethrough, the two fluids remain fluidly isolated from each other. Dual lumen tube 174 also provides an interface for a peristaltic pump contained within thermal control unit 822 to apply pressure to the fluids therein and pump them throughout cartridge 858, as well as back to the patient. That is, when thermal control unit 822 is adapted to be used with cartridges like cartridge 858, cartridge interface 156 includes a rotor with one or more shoes or rollers attached thereto that compress the flexible dual lumen tube 174 as they rotate, thereby pumping the fluids therein throughout cartridge 858 and back to the patient.

As shown in FIG. 9, cartridge 858 includes a temperature sensor region 176, a blood pressure sensor region 178, and an oxygenation level region 180. Each of these three regions provides areas for corresponding sensors positioned within cartridge interface 156 to take readings. For example, the blood temperature sensor region 176 includes, in some embodiments, a thin metallic foil, or other thin thermal conductor, that includes an internal surface positioned in direct contact with the blood within cartridge 858. Its external surface is exposed and comes into direct contact with a temperature sensor positioned at an adjoining location within cartridge receptacle 154. The temperature sensor therefore measures a temperature of the thin metallic foil, or other thermal conductor. Because the foil, or other thermal conductor, is a good thermal conductor, its temperature is substantially equal to the temperature of the blood on the opposite side, and therefore provides an accurate proxy reading of the temperature of the patient's blood.

With respect to blood pressure sensor region 178, cartridge 858 includes a flexible wall in this region that is flexible enough to change position in response to the diastolic and/or systolic pressures created by the patient's circulatory system. Cartridge receptacle 154 includes a sensor that is positioned to come into contact with blood pressures sensor region 178 and to detect the movement of the flexible wall within blood pressure sensor region 178. This movement is converted into a blood pressure reading either by controller 878, or a separate controller dedicated to convert the movements detected at region 178 into blood pressure readings. In some cases, controller 878 (or another controller) is adapted to filter out frequencies that are outside the normal blood pressure range. Mechanical filters may also be coupled to the blood pressure sensor within cartridge receptacle 154. Still further, controller 878 (or another controller) can be configured to take into account pressure changes caused by the peristaltic pump that squeezes dual lumen tube 174, and/or blood pressure region 178 can be positioned within cartridge 858 at a location that is substantially pressure-isolated from the actions of the peristaltic pump. Still other factors may be used by controller 878 to calculate the blood pressure of the patient, including, but not limited to, utilizing pre-stored data empirically gathered from patients whose blood pressure was measured via both sensor region 178 and via measurement (e.g. a conventional sphygmomanometer).

Oxygenation region 180 provides a window into cartridge 858 that allows an oxygenation sensor to determine the level of oxygenation of the patient's blood. In some embodiments, the window is translucent or semi-translucent. In some embodiments, an oxygenation sensor of the type disclosed in any of the following commonly assigned U.S. patent applications may be used: U.S. patent application Ser. No. 15/185,347 filed Jun. 17, 2016, by inventors Marko Kostic and entitled TISSUE MONITORING APPARATUS AND SYSTEM, and U.S. patent application Ser. No. 15/200,818 filed Jul. 1, 2016, by inventors Marko Kostic et al. and entitled SYSTEMS AND METHODS FOR STROKE DETECTION, the complete disclosures of both of which are incorporated herein by reference. Other types of oxygenation sensors may also be used.

FIG. 10 illustrates one suitable arrangement for the internal flow channels within cartridge 858. Other arrangements of channels may also or alternatively be used. The particular arrangement shown in FIG. 10 is designed for a cartridge in which the heater and cooler are contained internally within the cartridge. In other embodiments, the heater and/or cooler may be maintained external to the cartridge, such as in cartridge interface 156, wherein direct contact between the heater/cooler and the cartridge is established when the cartridge is inserted into receptacle 154.

Cartridge 858 includes a blood flow channel 182, a fluid flow channel 184, and a plurality of valve 186a-f. Blood flow channel 182 is in fluid communication with blood inlet and outlet ports 168 and 170. Fluid flow channel 184 is in fluid communication with fluid outlet and inlet ports 172 and 173. Blood flow channel 182 and fluid flow channel 184 are fluidly isolated from each other such that the patient's blood never mixes with the fluid when cartridge 858 is used with a patient. Blood entering cartridge 858 first flows through blood flow channel 182 past a first valve 186a. First valve 186a is opened when it is desired to allow blood to bypass both a chiller 188 and a heater 190. First valve 186a, as with all valves, is capable of being fully opened, fully closed, and a virtually infinite number of positions in-between, thereby allowing the amount of blood bypassing chiller 188 and heater 190 to be precisely controlled. Further, all of the valves 186 are controlled by a controller (not shown) internal to cartridge 858. This controller takes its instructions, in some embodiments, from controller 878, which may communicate with it wirelessly or via a wired interface that is established when cartridge 858 is inserted into receptacle 154.

After flowing past first valve 186a, the blood flow next encounters a second valve 186b. Second valve 186b controls the amount of blood that is passed to chiller 188 and what amount bypasses chiller 188 and proceeds directly to heater 190. The blood that flows past second valve 186b encounters a first pressure valve 192a that opens when a minimum amount of pressure is applied thereto. The blood therefore only enters chiller 188 when it experiences sufficient pressure. After exiting chiller 188, the chilled blood is rejoined by any blood that bypassed chiller 188 (via second valve 186b) before entering heater 190. After passing through heater 190, the blood is rejoined by any blood that bypassed heater 190 (via first valve 186a). After that, the blood flows to blood outlet port 170 and exits cartridge 858.

Fluid entering cartridge 858 first flows along fluid channel 184 until it encounters third valve 186c. Third valve 186c allows some, all, or none of the entering fluid to bypass both chiller 188 and heater 190. After passing by third valve 186c, the fluid encounters fourth valve 186d. Fourth valve 186d, when open, allows the fluid to bypass chiller 188, but not heater 190. The fluid that does not bypass chiller 188 encounters a second pressure valve 192b and only enters chiller 188 when sufficient pressure is built up in the fluid. After the fluid passes through chiller 188, it rejoins any fluid that bypassed chiller 188 via fourth valve 186d. The fluid is then pumped to heater 190. After passing through heater 190, the fluid rejoins any fluid that bypassed both chiller 188 and heater 190 via third valve 186c. From there, the fluid flows to fluid outlet port 172 and exits cartridge 858.

It will be understood that chiller 188 and heater 190 are independently controllable and that controller 878 (or another controller under its control) may only activate a single one of these two at certain times, or it may simultaneously activate both of these. Regardless of whether only a single one is currently activated or they are both simultaneously activated together, controller 878 is able to independently control the temperatures of the blood and fluid exiting cartridge 858 by selectively routing the blood and fluid in different proportions via valves 186a-d. In this manner, the blood exiting cartridge 858 may have a temperature that is different from the temperature of the fluid exiting cartridge 858.

Although cartridge 858 has been described herein as having a single dual lumen tube 174, it will be understood that cartridge 858 may be modified to include separate single lumen tubes for the blood and fluid. In this manner, different flow rates may be more easily achieved, although the use of valves 186a-d and 192a-b may be used to achieve independent flow rates for the blood and fluid. Alternatively, a flow control valve or restrictor may be placed in-line with each of the blood and fluid flow channels 182 and 184. Additional sensor regions and/or sensors may also be incorporated into cartridge 858, such as, but not limited to, a pulse sensor, one or more flow sensors, additional temperature sensors (e.g. to measure the temperature of the blood and/or fluid both before and after being temperature treated by chiller 188 and heater 190. Still other variations may be implemented.

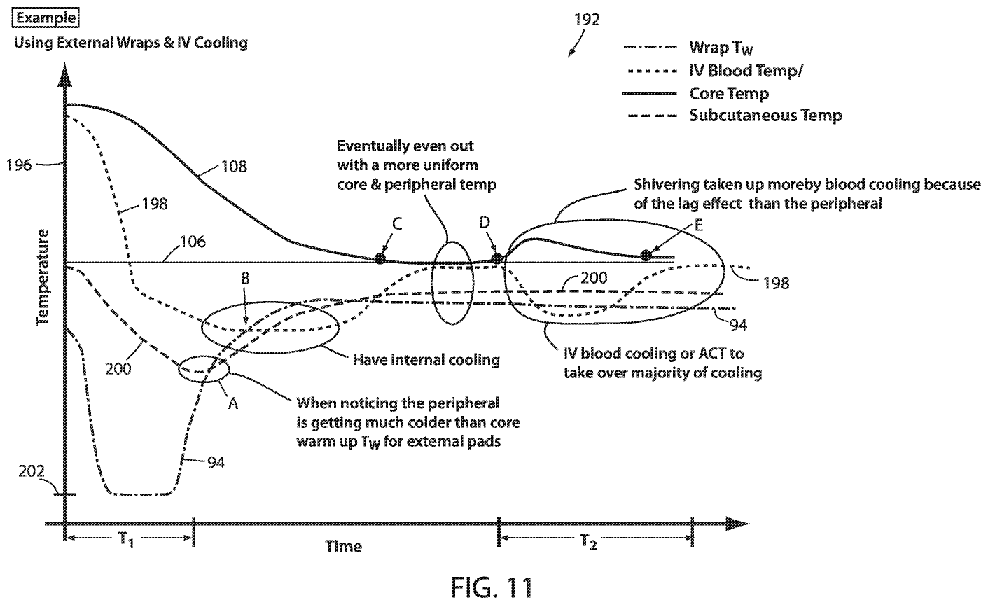

FIG. 11 illustrates a sample graph 192 of various temperatures that may result when thermal control unit 822 is used in a typical fashion to control a patient's blood temperature using cartridge 158 and/or 858. Graph 192 includes an X-axis 194 that represents time and a Y-axis 196 that represents temperature. Graph 192 includes four temperatures: a patient core temperature 108, a patient's blood temperature 198, a fluid temperature 94, and a patient peripheral temperature 200. Core temperature 108 is measured by a patient core temperature probe, such as probe 90. The blood temperature 198 is measured by a temperature sensor positioned in or adjacent to cartridge 158 or 858. The fluid temperature 94 is measured by inlet temperature sensors 140, outlet temperature sensors 138, a fluid temperature sensor (not shown) that is integrated into the cartridge 158 or 858, and/or a fluid temperature sensor (not shown) that is integrated into cartridge receptacle 154 and interacts with a fluid temperature sensor region similar to blood temperature sensor region 176. The patient peripheral temperature 200 is measured by a peripheral temperature probe 116 and/or inferred from the difference in the temperature between the fluid delivered to the thermal pads 24 and the fluid returning from the thermal pads 24.

During an initial period of time T1, the patient is cooled toward a target temperature 106 by cooling the patient's blood and by cooling the fluid circulating in thermal pads 24. As shown in FIG. 11, the fluid and the patient's blood are controlled independently (i.e. they are controlled to different target temperatures for much of the time period of FIG. 11). During time period T1, the fluid circulating in pads 24 is cooled to a predetermined minimum temperature 202 and the blood is cooled toward a target temperature that is warmer than the fluid temperature. Controller 878 monitors the difference between the patient's peripheral temperature 200 and the patient's blood temperature 198 during time period T1. In region A, controller 878 detects that the patients' peripheral temperature has begun to increase its rate of temperature decrease with respect to the patient's blood temperature 198. That is, the difference between these two temperatures begins to increase in region A, and the increase is caused by the peripheral temperature 200 falling more quickly than the blood temperature. As a result of this, controller 878 begins to warm the fluid circulating in the thermal pads 24. This warming occurs toward the end of time period T1 and is done, in at least some embodiments, to reduce the thermal stress on the patient by limiting the temperature gradient within the patient's body between his or her peripheral tissues and his or her blood.

After starting to warm the fluid in the thermal pads 24 toward the end of time period T1, the temperature of the fluid eventually is warmed to a temperature equal to the patient's blood temperature 198 at point B. In the region after point B, the cooling of the patient's blood becomes the primary means of cooling the patient. This continues until approximately point C where the patient's core temperature 108 reaches, or nearly reaches, the patient target temperature 106. At or near point C, controller 878 warms the blood until the blood temperature 198 is nearly the same as the patient's core temperature 108. From point C onward, both the patient's blood temperature 198 and the fluid temperature 94 of the thermal pads 24 are maintained at substantially constant temperatures. This generally keeps the patient's core temperature 108 steady at the patient target temperature 106.

FIG. 11 illustrates the scenario where the patient starts to shiver at about point D. This shivering tends to increase the core temperature 108 of the patient. Thermal control unit 822 is adapted to combat this rise of temperature due to shivering by primarily lowering the temperature of the patient's blood rather than by lowering the temperature of the fluid in the thermal pads 24. This shivering occurs in FIG. 11 during time period T2 and it can be seen that during this time period the patient's peripheral temperature 200 and the fluid temperature 94 remain generally constant while the patient's blood temperature 198 is lowered by thermal control unit 822. The lowering of the patient's blood temperature 198 during this shivering episode combats the rise in the patient's core temperature 108 and continues for as long as it takes to reduce the patient's core temperature 108 back to the target temperature 106. After reaching the target temperature 106 again at point E, controller 878 adjusts the patient's blood temperature 198 back to a temperature similar to what it was immediately prior to point D. The patient's core temperature 108 is thereafter maintained at target temperature 106 until the caregiver adjusts the target temperature 106 and/or shivering, or some other temperature-changing event, occurs.

In some embodiments, controller 878 detects shivering of the patient automatically and reacts in the manner shown in FIG. 11. Methods for automatically detecting a patient's shivering are disclosed in commonly assigned U.S. patent application Ser. No. 62/425,813 filed Nov. 23, 2016, by inventors Gregory Taylor et al. and entitled THERMAL SYSTEM, the complete disclosure of which is incorporated herein by reference. Other manners of detecting shivering may also be used, including a manual indication by a caregiver to controller 878 (via user interface 82) that shivering is occurring. Any of the other controllers and thermal control units discussed herein may also be modified to include automatic shivering detection structures and algorithms, including, but not limited to, those disclosed in the aforementioned '813 application.

It will be understood that, although cartridge 858 has been described herein as being used in conjunction with thermal control unit 822, cartridge 858 may be used with thermal control units that are different from thermal control unit 822. For example, cartridge 858 may be used with a thermal control unit that does not include outlet ports 58 and inlet ports 60, or any of the structures associated therewith (e.g. manifold 62, fluid circulation channel 36, etc.). This is because cartridge 858 includes its own fluid flow channel 184 that is able to supply temperature-controlled fluid to thermal pads 24. However, if cartridge 858 is used with a thermal control unit having fluid outlet ports 58 and fluid inlet ports 60, such as thermal control unit 822, those outlet ports and inlet ports 58 and 60 may be left unused during thermal treatment using cartridge 858, or one or more of those outlet ports and inlet ports 58 and 60 may be used to supply temperature-controlled fluid to one or more of the thermal pads 24. When outlet ports 58 and inlet ports 60 are used, the temperature-controlled fluid supplied to thermal pads 24 may come from both cartridge 858 and from one or more outlets 58. Still further, the thermal control unit used with cartridge 858 (including thermal control unit 822) may be configured to allow a user to select whether the thermal therapy to be applied to the patient will utilize both temperature control of the patient's blood and temperature control of the fluid supplied to thermal pads 24. The caregiver can therefore carry out thermal therapy using only temperature control of the patient's blood, or only temperature control of the fluid supplied to thermal pads 24, or both. Further, this selection can be carried out at any time during the thermal treatment of the patient.

It will also be understood by those skilled in the art that any of the features, functions, and/or structures from any of the thermal control units 22, 222, 422, and/or 822, as well as any of the features, functions, and/or structures of thermal control system 20, 220, 420, 620, and/or 820 may be incorporated into any of the other thermal control units and/or thermal control systems. Thus, for example, thermal control unit 822 may utilize one or more patient's non-temperature parameters (e.g. BMI) when controlling the patient's blood temperature and/or the temperature of the fluid supplied to the thermal pads 24. Further, the manner in which thermal control unit 822 utilizes the patient non-temperature parameter(s) may be in any of the manners discussed previously with respect to the other thermal control unit s. As another example, any of the thermal control units and/or thermal control systems disclosed herein may be configured to infer a patient peripheral temperature from the rate of heat exchange with the patient (and other factors, such as, but not limited to, patient weight) and to use the inferred temperature in any of the manners discussed herein in which peripheral temperature probe 116 is used.

As still another example, any of the thermal control units discussed herein may be configured to calculate a Q value for the auxiliary fluid delivered to the patient by measuring the temperature of the fluid when delivered to the patient and the temperature of the fluid coming from the patient, and then multiplying the temperature difference by the specific heat of the fluid and its flow rate. This includes both the patient's blood and other fluids. In the case of the temperature-controlled air, the Q value can be calculated by measuring the temperature of the air and assuming the air exits from the patient at the temperature of the patient's core. Still other combinations of features, functions, and/or other structures may be implemented.

It will also be understood that any of the thermal control units disclosed herein may be modified to additionally operate in conjunction with one or more auxiliary sensors used to sense one or more non-temperature patient parameters. When so modified, any of the thermal control units disclosed herein may utilize the auxiliary sensors in any of the manners, and using any of the structures and/or algorithms, disclosed in commonly assigned U.S. patent application Ser. No. 62/610,327 filed Dec. 26, 2017, by inventors Gregory S. Taylor et al. and entitled THERMAL SYSTEM WITH PATIENT SENSOR(S), the complete disclosure of which is incorporated herein by reference.

Still further, it will be understood that any of the thermal control units disclosed herein may incorporate any of the graphical user interface and/or other concepts disclosed in commonly assigned U.S. patent application Ser. No. 62/610,362 filed Dec. 26, 2017, by inventor Gregory S. Taylor and entitled THERMAL SYSTEM WITH GRAPHICAL USER INTERFACE, the complete disclosure of which has been incorporated herein by reference. Any of the thermal control units disclosed herein may also or alternatively be modified to incorporate any of the temperature overshoot reduction methods, structures, and/or algorithms disclosed in commonly assigned U.S. patent application Ser. No. 62/610,319 filed Dec. 26, 2017, by inventors Gregory Taylor et al. and entitled THERMAL SYSTEM WITH OVERSHOOT REDUCTION, the complete disclosure of which is incorporated herein by reference.

Various additional alterations and changes beyond those already mentioned herein can also be made to the above-described embodiments. This disclosure is presented for illustrative purposes and should not be interpreted as an exhaustive description of all embodiments or to limit the scope of the claims to the specific elements illustrated or described in connection with these embodiments. For example, and without limitation, any individual element(s) of the described embodiments may be replaced by alternative elements that provide substantially similar functionality or otherwise provide adequate operation. This includes, for example, presently known alternative elements, such as those that might be currently known to one skilled in the art, and alternative elements that may be developed in the future, such as those that one skilled in the art might, upon development, recognize as an alternative. Any reference to claim elements in the singular, for example, using the articles “a,” “an,” “the” or “said,” is not to be construed as limiting the element to the singular.

1. A thermal control unit for controlling a patient's temperature, the thermal control unit comprising:
a fluid outlet adapted to fluidly couple to a fluid supply line;
a fluid inlet adapted to fluidly couple to a fluid return line;
a fluid circulation path coupled to the fluid inlet and the fluid outlet;
a pump for circulating fluid through the fluid circulation path from the fluid inlet to the fluid outlet;
a blood inlet adapted to receive blood from the patient;
a blood outlet adapted to return blood to the patient;
a blood circulation path coupled to the blood outlet and the blood inlet;
a heat exchange subsystem for adding and removing heat from the fluid and the blood; and
a controller in communication with the heat exchange subsystem and adapted to control a temperature of the fluid and a temperature of the blood in order to bring the patient to a target patient temperature.

2. The thermal control unit of claim 1 wherein the heat exchange subsystem comprises a first heat exchanger adapted to add or remove heat from the fluid circulating through the fluid circulation path and a second heat exchanger adapted to add or remove heat from the blood circulating through the blood circulation path, and wherein the controller is adapted to independently control the fluid temperature to a target fluid temperature and the blood temperature to a target blood temperature, and the target fluid temperature and the target blood temperature may vary from each other.

3. (canceled)

4. The thermal control unit of claim 2 further comprising:
a core temperature probe port adapted to receive a core temperature probe for measuring a core temperature of the patient; and
a peripheral temperature probe port adapted to receive a peripheral temperature probe for measuring a peripheral temperature of the patient;
wherein the controller is adapted to control the temperature of both the fluid and the blood based on temperature readings received from both the core temperature probe port and the peripheral temperature probe port.

5. The thermal control unit of claim 2 wherein the controller is adapted to control the second heat exchanger based upon a temperature of the blood in the blood circulation path and the controller is adapted to control the first heat exchanger based upon a temperature of both the blood and the fluid in the fluid circulating path.

6. (canceled)

7. The thermal control unit of claim 2 wherein the fluid circulation path and blood circulation path are both defined within a cartridge adapted to be inserted into and out of a chamber defined in the thermal control unit, and wherein the first and second heat exchangers are fluidly isolated from the cartridge and remain within the thermal control unit when the cartridge is removed.

8. (canceled)

9. The thermal control unit of claim 2 wherein the controller is adapted to infer a patient peripheral temperature at a location adjacent a thermal pad based upon a heat transfer rate between the patient and the thermal pad, the thermal pad being fluidly coupled to the fluid outlet and fluid inlet, and the controller being further adapted to control the first and second heat exchangers based on the inferred patient peripheral temperature.

10. The thermal control unit of claim 2 further including a user interface adapted to receive a non-temperature patient parameter, wherein the controller is further adapted to control the first and second heat exchangers based partially on the non-temperature patient parameter, and wherein the non-temperature patient parameter is one of a body mass index (BMI), a body surface area (BSA), a patient weight, or a patient height.

11. (canceled)

12. The thermal control unit of claim 2 further comprising a first intravenous needle coupled to the blood inlet and a second intravenous needle coupled to the blood outlet, each of the first and second intravenous needles being adapted to be inserted into a peripheral vein of the patient.

13. The thermal control unit of claim 2 further comprising a sensor positioned within the thermal control unit, the sensor adapted to detect a vital sign of the patient from the blood flowing through the blood circulation path, wherein the vital sign is an oxygenation level of the patient's blood.

14. (canceled)

15. A thermal control unit for controlling a patient's temperature, the thermal control unit comprising:
a fluid outlet adapted to fluidly couple to a fluid supply line of a thermal pad, the thermal pad adapted to be wrapped around a portion of the patient's body;
a fluid inlet adapted to fluidly couple to a fluid return line of the thermal pad;
a circulation channel coupled to the fluid outlet and the fluid inlet;
a pump for circulating fluid through the circulation channel from the fluid inlet to the fluid outlet;
a heat exchanger adapted to add or remove heat from the fluid circulating in the circulation channel;
a fluid temperature sensor adapted to sense a temperature of the fluid;
a patient temperature probe port adapted to receive patient core temperature readings from a patient temperature probe;
a user interface adapted to receive a non-temperature patient parameter; and
a controller in communication with the patient temperature probe port, the pump, the fluid temperature sensor, and the user interface, the controller adapted to control the heat exchanger based on both the patient core temperature readings and the non-temperature patient parameter.

16. The thermal control unit of claim 15 wherein the controller is adapted to control the heat exchanger in order to automatically bring a temperature of the patient to a target patient temperature, and wherein the non-temperature patient parameter is one of a body mass index (BMI), a body surface area (BSA), a patient weight, or a patient height.

17. (canceled)

18. The thermal control unit of claim 15 further comprising a patient peripheral temperature port adapted to receive patient peripheral temperature readings from a peripheral patient temperature sensors adapted to measure a peripheral temperature of the patient, the controller further adapted to control the heat exchanger based on differences between the patient core temperature readings and the patient peripheral temperature readings.

19. (canceled)

20. The thermal control unit of claim 15 wherein the controller is adapted to infer a patient peripheral temperature at a location adjacent the thermal pad based upon a heat transfer rate between the patient and the thermal pad, and the controller is further adapted to control the heat exchanger based on the inferred patient peripheral temperature.

21. The thermal control unit of claim 15 further comprising:
a second fluid outlet adapted to couple to a fluid supply line of an auxiliary thermal therapy device, the auxiliary thermal therapy device being of a type other than a thermal pad, the auxiliary thermal therapy device being adapted to add or remove heat from the patient; and
a second fluid inlet adapted to couple to a fluid return line of the auxiliary thermal therapy device;
wherein the auxiliary thermal therapy device includes a fluid passageway positioned in the patient's esophagus and the fluid passageway is adapted to receive circulating fluid from the second fluid outlet.

22. (canceled)

23. The thermal control unit of claim 21 wherein the second fluid outlet and second fluid inlet are fluidly coupled to each other via a second circulation channel, the second circulation channel is fluidly isolated from the circulation channel, the second circulation channel is adapted to receive blood from the patient, and the auxiliary thermal therapy device includes a first intravenous needle coupled to the second fluid outlet and a second intravenous needle coupled to the second fluid inlet, wherein each of the first and second intravenous needles are adapted to be inserted into a peripheral vein of the patient.

24. (canceled)

25. The thermal control unit of claim 15 further comprising a transceiver adapted to communicate with an auxiliary thermal therapy device, the auxiliary thermal therapy device adapted to supply heat to, and remove heat from, the patient by delivering temperature-controlled air to be breathed in by the patient.

26. (canceled)

27. A thermal control unit for controlling a patient's temperature, the thermal control unit comprising:
a fluid outlet adapted to fluidly couple to a fluid supply line of a thermal pad, the thermal pad adapted to be wrapped around a portion of the patient's body;
a fluid inlet adapted to fluidly couple to a fluid return line of the thermal pad;
a circulation channel coupled to the fluid outlet and the fluid inlet;
a pump for circulating fluid through the circulation channel from the fluid inlet to the fluid outlet;
a heat exchanger adapted to add or remove heat from the fluid circulating in the circulation channel;
a fluid temperature sensor adapted to sense a temperature of the fluid;
a patient core temperature probe port adapted to receive patient core temperature readings from a patient temperature probe;
a patient peripheral temperature probe port adapted to receive patient peripheral temperature readings from a peripheral temperature sensor; and
a controller in communication with the patient core temperature probe port, the patient peripheral temperature probe port, the pump, and the fluid temperature sensor, the controller adapted to determine differences between the patient core temperature readings and the patient peripheral temperature readings and to automatically control a temperature of the fluid in order to prevent the differences from exceeding a predetermined maximum.

28. The thermal control unit of claim 27 wherein the controller is adapted to control the heat exchanger in order to automatically bring the patient core temperature readings to a target patient temperature, and wherein the controller is adapted to control the heat exchanger based on at least one of the following: a body mass index (BMI) of the patient, a body surface area (BSA) of the patient, a patient weight, or a patient height.

29. (canceled)

30. The thermal control unit of claim 28 further comprising a transceiver adapted to communicate with an auxiliary thermal therapy device, the auxiliary thermal therapy device adapted to supply heat to, and remove heat from, the patient.

31. (canceled)

32. The thermal control unit of claim 30 wherein the auxiliary thermal therapy device is adapted to deliver temperature-controlled air to be breathed in by the patient.

33-54. (canceled)
